(19) United States
(12) Patent Application Publication
(10) Pub. No.: US 2021/0114988 A1
(43) Pub. Date: Apr. 22, 2021

(54) PHOTOELECTRIC CONVERSION ELEMENT

(71) Applicant: SONY CORPORATION, Tokyo (JP)

(72) Inventors: Hajime KOBAYASHI, Tokyo (JP); Shinnosuke HATTORI, Tokyo (JP)

(73) Assignee: SONY CORPORATION, Tokyo (JP)

(21) Appl. No.: 17/257,104

(22) PCT Filed: Jun. 6, 2019
(86) PCT No.: PCT/JP2019/022599

§ 371 Date: Dec. 30, 2020

(30) Foreign Application Priority Data
Jul. 9, 2018 (JP) 2018-129974

(51) Int. Cl. C07D 221/14; H01L 27/146; H01L 27/30; H01L 51/44; C07D 221/18; C07D 471/04; C07C 15/62

(52) U.S. Cl. CPC C07D 221/14; H01L 27/14643; H01L 27/307; C07C 2603/24; C07D 221/18; C07D 471/04; C07C 15/62; H01L 51/441

(57) ABSTRACT

To provide a photoelectric conversion element capable of improving afterimage characteristics. It includes a first electrode and a second electrode arranged to face each other, and a photoelectric conversion layer provided between the first electrode and the second electrode and including a first organic semiconductor material and a second organic semiconductor material, in which the first organic semiconductor material or the second organic semiconductor material includes an organic semiconductor having a D₃/Dₜₒₜ of 0.01 or more.

### TECHNICAL FIELD

The technology according to the present disclosure (present technology) relates to a photoelectric conversion element.

### BACKGROUND ART

In recent years, in a solid-state imaging element such as a CCD (Charge Coupled Device) image sensor and a CMOS (Complementary Metal Oxide Semiconductor) image sensor, a reduction of a pixel size is in progress. Thus, because the number of photons incident on a unit pixel is reduced, sensitivity is lowered and a decrease in an S/N ratio occurs. Furthermore, in a case where color filters formed by two-dimensionally arranging primary color filters of red, green, and blue for coloring are used, green light and blue light are absorbed by the color filters in red pixels, resulting in a decrease in the sensitivity. Moreover, since interpolation processing is performed between pixels when generating each color signal, so-called false colors are generated.

Therefore, for example, Patent Literature 1 discloses a solid-state imaging element in which, for example, an organic photoelectric conversion section that detects green light and generates a signal charge corresponding thereto, and a photodiode (inorganic photoelectric conversion section) that detects each of red light and blue light are provided in one pixel, and a signal of three colors is obtained in one pixel, thereby improving the decrease in the sensitivity. A photoelectric conversion layer constituting the organic photoelectric conversion section in the solid-state imaging element has a bulk hetero structure in which a p-type organic semiconductor material and an n-type organic semiconductor material are randomly mixed.

One of important characteristics of the solid-state imaging element is afterimage characteristics. In order to obtain excellent afterimage characteristics, it is necessary to keep a carrier mobility of an entire bulk hetero layer high. Often, a material used as a hole transport material is easy to crystallize, is single crystal, and have high mobility.

### CITATION LIST

### Patent Literature

Patent Literature 1: Japanese Patent Application Laid-open No. 2003-332551
Non-Patent Literature 1: 1. H. Kobayashi, R. Shirasawa, M. Nakamoto, S. Hattori, and S. Tomiya, Appl. Phys. Lett. 111, 033301 (2017)

### DISCLOSURE OF INVENTION

### Technical Problem

However, when such a material that is single crystal and has high hole mobility is used in a bulk hetero layer, the mobility may be nevertheless extremely lowered. When the mobility is lowered, a longer time is required for a charge generated at a charge separation interface to reach an electrode and therefore there is a problem that the afterimage characteristics of the photoelectric conversion element is lowered.

The present technology is made in view of such problem, and an object thereof is to provide a photoelectric conversion element capable of improving the afterimage characteristics.

### Solution to Problem

One embodiment of the present technology is a photoelectric conversion element including a first electrode and a second electrode arranged to face each other; and a photoelectric conversion layer provided between the first electrode and the second electrode and including a first organic semiconductor material and a second organic semiconductor material, in which the first organic semiconductor material or the second organic semiconductor material includes an organic semiconductor having D₃/Dₜₒₜ of 0.01 or more. According to this embodiment, the first organic semiconductor material or the second organic semiconductor material including the organic semiconductor has high hole mobility even in a bulk hetero layer. Thus, it is possible to provide a photoelectric conversion element capable of improving afterimage characteristics.

### BRIEF DESCRIPTION OF DRAWINGS

FIG. 1 is a cross-sectional view showing a configuration example of a photoelectric conversion element according to an embodiment of the present technology.

FIG. 2 is a cross-sectional view showing another configuration example of a photoelectric conversion element according to an embodiment of the present technology.

FIG. 3 is a diagram showing an anisotropy of a diffusion coefficient in microcrystal.

FIG. 4 is a diagram showing a modeling of a bulk hetero layer used in a coarse-grained kMC method.

FIG. 5 is a graph showing a relationship between a composition ratio (horizontal axis) and mobility (vertical axis) of pentacene.

FIG. 6 is a graph showing a relationship between a composition ratio (horizontal axis) and mobility (vertical axis) of rubrene.

FIG. 7 is a graph showing a relationship between a composition ratio (horizontal axis) and mobility (vertical axis) of C₈-BTBT.

FIG. 8 is a graph showing a relationship between a compositional ratio (horizontal axis) and mobility (vertical axis) of DPh-BTBT.

FIG. 9 is a graph showing a relationship between a composition ratio (horizontal axis) and mobility (vertical axis) of α-QD.

FIG. 10 is a graph showing a relationship between a composition ratio (horizontal axis) and mobility (vertical axis) of β-QD.

FIG. 11 is a graph showing a relationship between a composition ratio (horizontal axis) and mobility (vertical axis) of γ-QD.

FIG. 12 is a graph showing a relationship between a composition ratio (horizontal axis) and mobility (vertical axis) of each material.

FIG. 13 is a graph showing a relationship between D₃/Dₜₒₜ (vertical axis) and P_{c} (horizontal axis).

FIG. 14 is a block diagram showing an example of a schematic configuration of an imaging element.

FIG. 15 is a block diagram showing an example of a schematic configuration of an electronic apparatus.

FIG. 16 is a view depicting an example of a schematic configuration of an endoscopic surgery system.

FIG. 17 is a block diagram depicting an example of a functional configuration of a camera head and a camera control unit (CCU).

FIG. 18 is a block diagram depicting an example of schematic configuration of a vehicle control system.

FIG. 19 is a diagram of assistance in explaining an example of installation positions of an outside-vehicle information detecting section and an imaging section.

### MODE(S) FOR CARRYING OUT THE INVENTION

Embodiments of the present technology will be described below with reference to the drawings. In the description of the drawings referred to in the following description, the same or similar parts are denoted by the same or similar reference numerals. It should be noted, however, that the drawings are schematic, and that the relationship between a thickness and plane dimensions, the ratio of the thickness of each layer, etc., are different from the actual ones. Therefore, the specific thickness and dimensions should be determined by referring to the following description. Moreover, it is needless to say that the drawings also include portions having different dimensional relationships and ratios from each other. In the following description, definitions of a vertical direction and the like are merely definitions for convenience of description and do not limit the technical idea of the present technology. For example, it is needless to say that when the object is rotated by 90 degrees and observed, the up and down are converted to the left and right and read, and when the object is rotated by 180 degrees and observed, the up and down are inverted and read.

(Photoelectric Conversion Element)

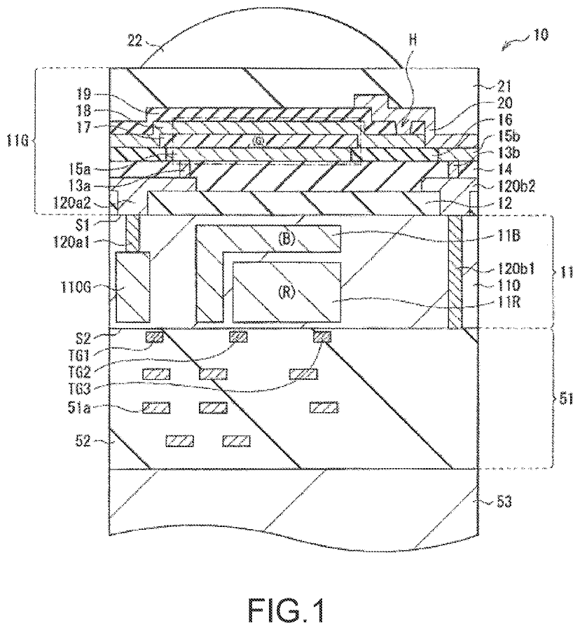

FIG. 1 is a cross-sectional view showing a configuration example of a photoelectric conversion element according to an embodiment of the present technology. A photoelectric conversion element 10 according to an embodiment of the present technology constitutes one pixel (unit pixel P) in a solid-state imaging element such as a CCD image sensor or a CMOS image sensor, for example. In the photoelectric conversion element 10, pixel transistors are formed on a surface side of a semiconductor substrate 11 (surface S2 opposite to light receiving surface (surface S1)), and a multilayer wiring layer 51 is included as well.

The photoelectric conversion element 10 of the present technology has a structure in which one organic photoelectric conversion section 11G for selectively detecting light of different wavelength regions and performing a photoelectric conversion and two inorganic photoelectric conversion sections 11B and 11R are laminated in the longitudinal direction, and the organic photoelectric conversion section 11G is configured to include three kinds of organic semiconductor materials.

The photoelectric conversion element 10 has a laminated structure of one organic photoelectric conversion section 11G and two inorganic photoelectric conversion sections 11B and 11R, so that each color signal of red (R), green (G), and blue (B) is acquired by one element. The organic photoelectric conversion section 11G is formed on a back surface (surface S1) of the semiconductor substrate 11, and the inorganic photoelectric conversion sections 11B, 11R are embedded formed in the semiconductor substrate 11. Hereinafter, the configuration of each section will be described.

The organic photoelectric conversion section 11G is an organic photoelectric conversion element that uses an organic semiconductor to absorb light in a selective wavelength region (here, green light) and to generate a pair of electronic holes. The organic photoelectric conversion section 11G has a configuration in which an organic photoelectric conversion layer 17 is sandwiched between a pair of electrodes for taking out the signal charge (lower electrode 15a, upper electrode 18). The lower electrode 15a (example of first electrode) and the upper electrode 18 (example of second electrode) are arranged to face each other. The lower electrode 15a and the upper electrode 18 are electrically connected to conductive plugs 120a1 and 120b1 buried in the semiconductor substrate 11 via wiring layers 13a, 13b, and 15b and a contact metal layer 20.

Specifically, in the organic photoelectric conversion portion 11G, interlayer insulating films 12 and 14 are formed on the surface S1 of the semiconductor substrate 11, through holes are provided in regions of the interlayer insulating film 12 facing the respective conductive plugs 120a1 and 120b1, and conductive plugs 120a2 and 120b2 are buried in the through holes. In the interlayer insulating film 14, wiring layers 13a and 13b are buried in regions facing the respective conductive plugs 120a2 and 120b2. On the interlayer insulating film 14, the lower electrode 15a is provided, and the wiring layer 15b electrically separated from the lower electrode 15a by an insulating film 16 is provided as well. Of these, the organic photoelectric conversion layer 17 is formed on the lower electrode 15a, and the upper electrode 18 is formed so as to cover the organic photoelectric conversion layer 17. A protective layer 19 is formed on the upper electrode 18 so as to cover the surface thereof. A contact hole H is provided in a predetermined region of the protective layer 19, and a contact metal layer 20 that fills the contact hole H and extends to the upper surface of the wiring layer 15b is formed on the protective layer 19.

The conductive plug 120a2 functions as a connector together with the conductive plug 120a1. In addition, the conductive plug 120a2 forms a transmission path of a charge (electron) from the lower electrode 15a to a green storage layer 110G, which will be described later, together with the conductive plug 120a1 and the wiring layer 13a. The conductive plug 120b2 serves as a connector together with the conductive plug 120b1. In addition, the conductive plug 120b2 forms a discharge path of the charge (hole) from the upper electrode 18 together with the conductive plug 120b1, the wiring layer 13b, the wiring layer 15b, and the contact metal layer 20. The conductive plugs 120a2 and 120b2 are desirably formed of a laminated film of a metal material such as titanium (Ti), titanium nitride (TiN) and tungsten in order to also function as a light shielding film. In addition, by using such a laminated film, even when the conductive plugs 120a1 and 120b1 are formed as n-type or p-type semiconductor layers, contact with silicon can be ensured, which is desirable.

The interlayer insulating film 12 is desirably configured of an insulating film with a low interface state in order to reduce the interface state between the semiconductor substrate 11 (silicon layer 110) and the interlayer insulating film 12 and suppress a generation of a dark current from the interface between the silicon layer 110 and the interlayer insulating film 12 as well. As such an insulating film, for example, a laminated film of a hafnium oxide (HfO₂) film and a silicon oxide (SiO₂) film can be used. The interlayer insulating film 14 is formed of, for example, a single layer film of one of silicon oxide, silicon nitride, silicon oxynitride (SiON) and the like, or a laminated film of two or more thereof.

The insulating film 16 is formed of, for example, a single layer film of one of silicon oxide, silicon nitride, silicon oxynitride (SiON) and the like, or a laminated film of two or more thereof. The surface of the insulating film 16 is planarized, for example, and has a shape and a pattern substantially without a step with the lower electrode 15a. The insulating film 16 has a function of electrically separating the lower electrode 15a of each pixel when the photoelectric conversion element 10 is used as the unit pixel P of the solid-state imaging element.

The lower electrode 15a faces light receiving surfaces of the inorganic photoelectric conversion sections 11B and 11R formed in the semiconductor substrate 11 and is provided in a region covering these light receiving surfaces. The lower electrode 15a is made of a conductive film having light transmittance, and is made of, for example, indium tin oxide (ITO). However, other than the ITO, a tin oxide (SnO₂) based material to which a dopant is added or a zinc oxide based material obtained by adding a dopant to aluminum zinc oxide (ZnO) may be used as a constituent material of the lower electrode 15a. Examples of the zinc oxide-based material include aluminum zinc oxide (AZO) to which aluminum (Al) is added as a dopant, gallium zinc oxide (GZO) to which gallium (Ga) is added, and indium zinc oxide (IZO) to which indium (In) is added. Furthermore, in addition to this, CuI, InSbO₄, ZnMgO, CuInO₂, MgIN₂O₄, CdO, ZnSnO₃, and the like may be used. Incidentally, according to the present technology, since the signal charge (electron) is taken out from the lower electrode 15a, in the solid-state imaging element using the photoelectric conversion element 10 as the unit pixel P, the lower electrode 15a is formed by separating for each pixel.

Other layer (not shown) may be provided between the organic photoelectric conversion layer 17 and the lower electrode 15a and between the organic photoelectric conversion layer 17 and the upper electrode 18. For example, an undercoat film, a hole transport layer, an electron blocking film, the organic photoelectric conversion layer 17, a hole blocking film, a buffer film, an electron transport layer, and a work function adjusting film may be laminated in this order from a lower electrode 15a side.

The upper electrode 18 is formed of a conductive film having a light transmittance similar to that of the lower electrode 15a. In the solid-state imaging element using the photoelectric conversion element 10 as a pixel, the upper electrode 18 may be separated for each pixel, or may be formed as a common electrode to each pixel. A thickness of the upper electrode 18 is, for example, 10 nm or more and 200 nm or less.

The protective layer 19 is made of a material having light transmittance, and is, for example, a single layer film made of any one of silicon oxide, silicon nitride, silicon oxynitride, or the like, or a laminated layer film made of two or more thereof. A thickness of the protective layer 19 is, for example, 100 nm or more and 30000 nm or less.

The contact metal layer 20 is made of, for example, any one of titanium (Ti), tungsten (W), titanium nitride (TiN), aluminum (Al), or the like, or a laminated film made of two or more thereof. The upper electrode 18 and the protective layer 19 are provided so as to cover the organic photoelectric conversion layer 17, for example.

A planarization layer 21 is formed on the protective layer 19 and the contact metal layer 20 so as to cover the entire surface. On the planarization layer 21, an on-chip lens 22 (microlens) is provided. The on-chip lens 22 collects light incident from above on each light receiving surface of the organic photoelectric conversion section 11G, the inorganic photoelectric conversion sections 11B and 11R. In the present technology, since the multilayer wiring layer 51 is formed on a surface S2 side of the semiconductor substrate 11, each light receiving surface of the organic photoelectric conversion section 11G, the inorganic photoelectric conversion sections 11B and 11R can be arranged close to each other, and it is possible to reduce a deviation in sensitivity among colors generated depending on an F value of the on-chip lens 22.

In the semiconductor substrate 11, the inorganic photoelectric conversion portions 11B and 11R and the green storage layer 110G are buried and formed in a predetermined region of the n-type silicon (Si) layer 110, for example. The conductive plugs 120a1 and 120b1 are buried in the semiconductor substrate 11 to be transmission paths of a charge (electron or hole) from the organic photoelectric conversion section 11G. In the present technology, the back surface (surface S1) of the semiconductor substrate 11 is a light receiving surface. On a surface (surface S2) side of the semiconductor substrate 11, a plurality of pixel transistors corresponding to each of the organic photoelectric conversion section 11G, and the inorganic photoelectric conversion section 11B and 11R is formed, and a peripheral circuit made of a logic circuit or the like is formed as well.

The respective inorganic photoelectric conversion sections 11B and 11R are photodiodes each having a pn junction and are formed on an optical path in the semiconductor substrate 11 in the order of the inorganic photoelectric conversion section 11B and 11C from a surface S1 side. Of these, the inorganic photoelectric conversion section 11B selectively detects blue light, accumulates a signal charge corresponding to blue, and is formed to extend from a selective region along the surface S1 of the semiconductor substrate 11 toward a region near the interface between the multilayer wiring layer 51 and the semiconductor substrate 11, for example. The inorganic photoelectric conversion section 11R selectively detects red light, accumulates a signal charge corresponding to red light, and is formed over a region of a lower layer (surface S2 side) than the inorganic photoelectric conversion section 11B, for example. Note that blue (B) is a color corresponding to, for example, a wavelength region of 450 nm or more and 495 nm or less, and red (R) is a color corresponding to, for example, a wavelength region of 620 nm or more and 750 nm or less, and the inorganic photoelectric conversion sections 11B and 11R may be capable of detecting light in part or all of each of the wavelength regions, respectively.

On the surface (surface S2) side of the semiconductor substrate 11, a plurality of pixel transistors corresponding to each of the organic photoelectric conversion section 11G and the inorganic photoelectric conversion section 11B and 11R (including transfer transistors) is formed, and a peripheral circuit including a logic circuit or the like is formed as well. For example, on the surface S2 of the semiconductor substrate 11, the multilayer wiring layer 51 is formed. On the multilayer wiring layer 51, gate electrodes TG1, TG2, and TG3 of the pixel transistor and a plurality of wires 51a are arranged via an interlayer insulating film 52. Thus, in the photoelectric conversion element 10, the multilayer wiring layer 51 is formed on the side opposite to the light receiving surface, and it is possible to realize a so-called back-surface irradiation type solid-state imaging element. To the multilayer wiring layer 51, a support substrate 53 made, for example, of silicon (Si) is bonded.

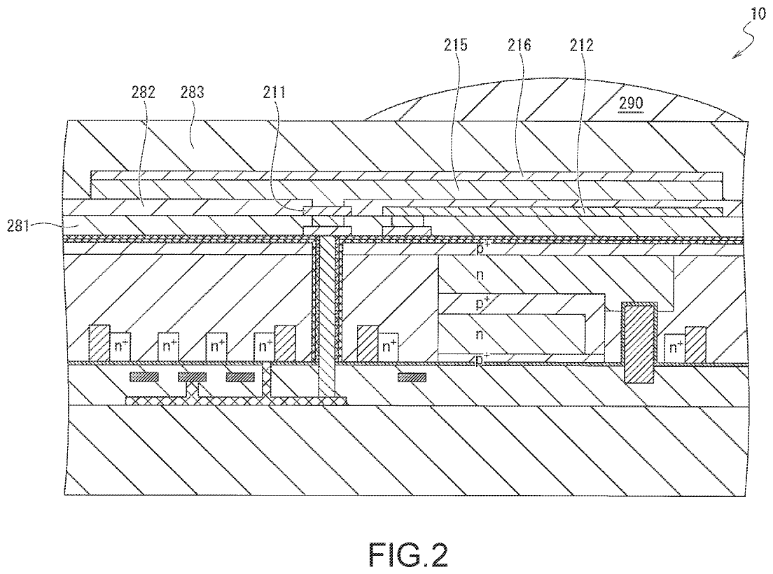

FIG. 2 is a cross-sectional view showing another configuration example of a photoelectric conversion element according to an embodiment of the present technology. As shown in FIG. 2, another configuration example of the photoelectric conversion element 10 according to the embodiment includes a photoelectric conversion section formed by laminating a first electrode 211, a photoelectric conversion layer 215, and a second electrode 216. The photoelectric conversion section further includes a charge storage electrode 212 arranged spaced apart from the first electrode 211 and arranged to face the photoelectric conversion layer 215 via an insulating layer 282. On the interlayer insulating layer 281, the first electrode 211 and the charge storage electrode 212 are formed spaced apart. The interlayer insulating layer 281 and the charge storage electrode 212 are covered by the insulating layer 282. The photoelectric conversion layer 215 is formed on the insulating layer 282, and the second electrode 216 is formed on the photoelectric conversion layer 215. A protective layer 283 is formed on the entire surface including the second electrode 216, and an on-chip micro lens 290 is provided on the protective layer 283. The other configuration is the same as the configuration example shown in FIG. 1. Incidentally, the photoelectric conversion layer 215 shown in FIG. 2 includes at least the first organic semiconductor material and the second organic semiconductor material. The specific configuration of the photoelectric conversion layer 215 is the same as that of the organic photoelectric conversion layer 17 described below.

(Organic Photoelectric Conversion Layer)

The organic photoelectric conversion layer 17 includes two kinds of the first organic semiconductor material and the second organic semiconductor material. Alternatively, the organic photoelectric conversion layer 17 may be configured to include three types of the first organic semiconductor material, the second organic semiconductor material, and a third organic semiconductor material. The organic photoelectric conversion layer 17 preferably includes one or both of a p-type semiconductor and an n-type semiconductor, and any of the two or three types of organic semiconductor materials is the p-type organic semiconductor material or the n-type organic semiconductor material. For example, the organic photoelectric conversion layer 17 has a bulk hetero structure in which the p-type organic semiconductor material and the n-type organic semiconductor material are randomly mixed. The organic photoelectric conversion layer 17 photoelectrically converts light in the selective wavelength region while transmitting light in another wavelength region, and in the present technology, for example, has a maximum absorption wavelength in a region of 450 nm to 650 nm.

Since the hole mainly conducts the p-type semiconductor after the photoelectric conversion, it is desirable that the p-type organic semiconductor material has high hole mobility.

In the present technology, the first organic semiconductor material or the second organic semiconductor material included in the organic photoelectric conversion element 17 includes an organic semiconductor having D₃/Dₜₒₜ of 0.01 or more. The D₃/Dₜₒₜ will be described in the following examples. The first organic semiconductor material or the second organic semiconductor material includes an organic semiconductor having P_{c} of 0.4 or less. The P_{c} will also be described in the following examples. Thus, the first organic semiconductor material or the second organic semiconductor material functioning as the p-type organic semiconductor material has the high hole mobility.

### (Specific Examples of Organic Semiconductors)

As the organic semiconductor included in the first organic semiconductor material or the second organic semiconductor material, any one or more of the compounds represented by the following formulae [Chemical Formula 1] can be cited.

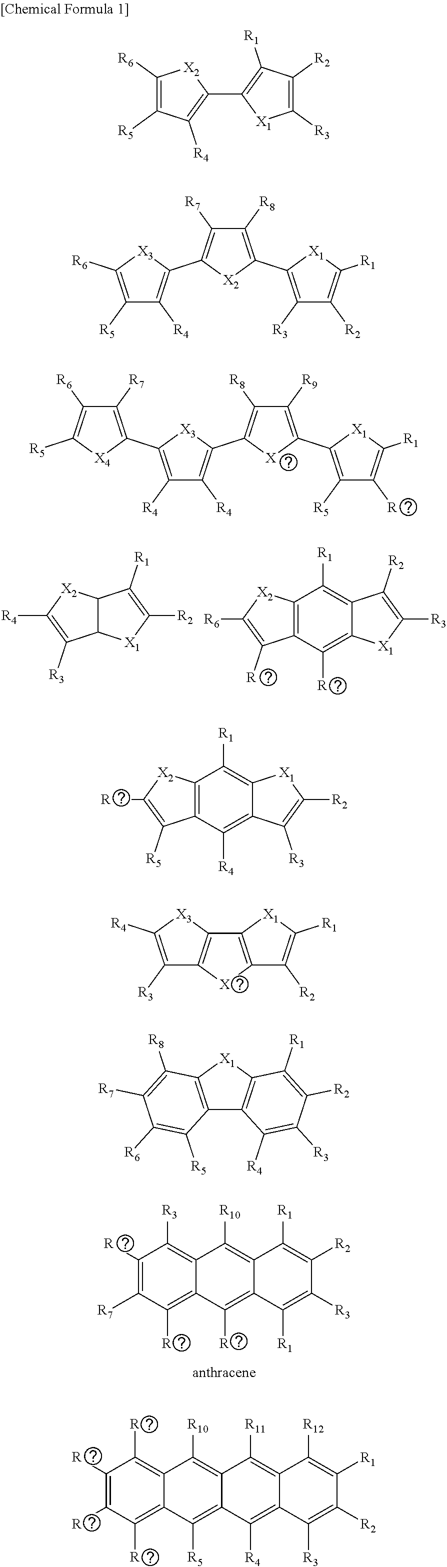

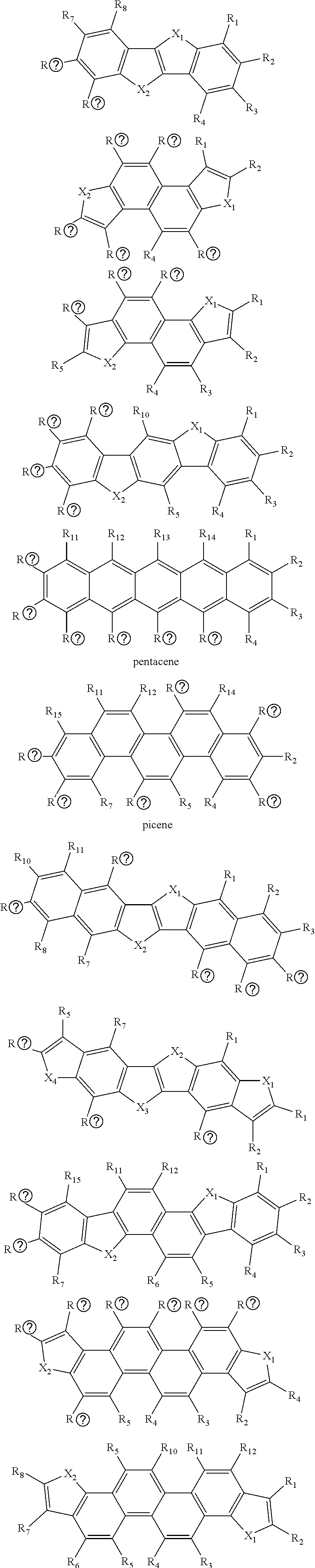

In each formula of [Chemical Formula 1], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group. Any adjacent Rs may be bonded to each other to form a fused aliphatic ring or a fused aromatic ring. X is each independently a heteroatom.

In addition, the organic semiconductor included in the first organic or second organic conductor material may be a compound represented by the (1) formula of [Chemical Formula 2] below.

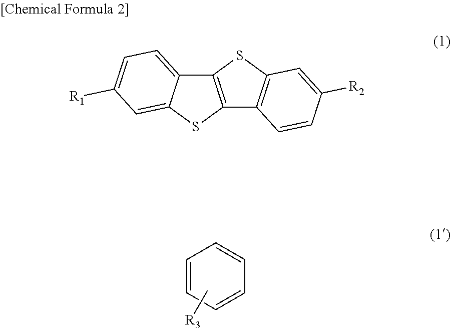

In Formula (1) of [Chemical Formula 2], R1 and R2 are each independently a hydrogen atom or a substituent represented by Formula (1)′. R3 is an aromatic ring group or an aromatic ring group having a substituent.)

In addition, the organic semiconductor contained in the first organic semiconductor material or the second organic semiconductor material may be a compound represented by the following formula of [Chemical Formula 3].

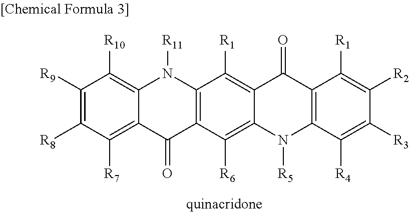

In the formula of [Chemical Formula 3], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group. Any adjacent Rs may be bonded to each other to form a fused aliphatic ring or a fused aromatic ring.

In addition, the organic semiconductor included in the first organic semiconductor material or the second organic semiconductor material may be any one or more of the compounds represented by the following formulae (4) of [Chemical Formula 4].

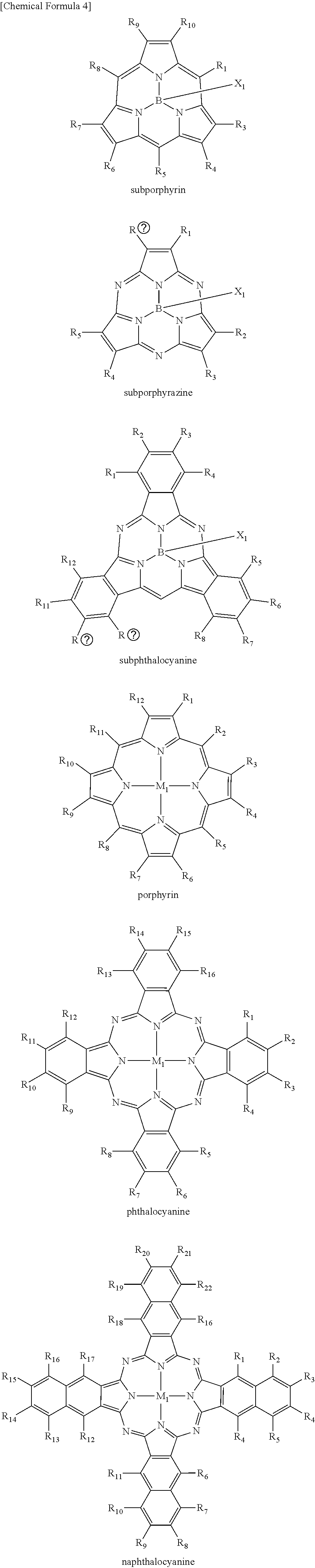

In each formula of [Chemical Formula 4], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group. Any adjacent Rs may be bonded to each other to form a fused aliphatic ring or a fused aromatic ring. X is an anionic group. M is a cationic group.

In addition, the organic semiconductor contained in the first organic semiconductor material or the second organic semiconductor material may be any one or more of the compounds represented by the following formulae (5) of [Chemical Formula 5].

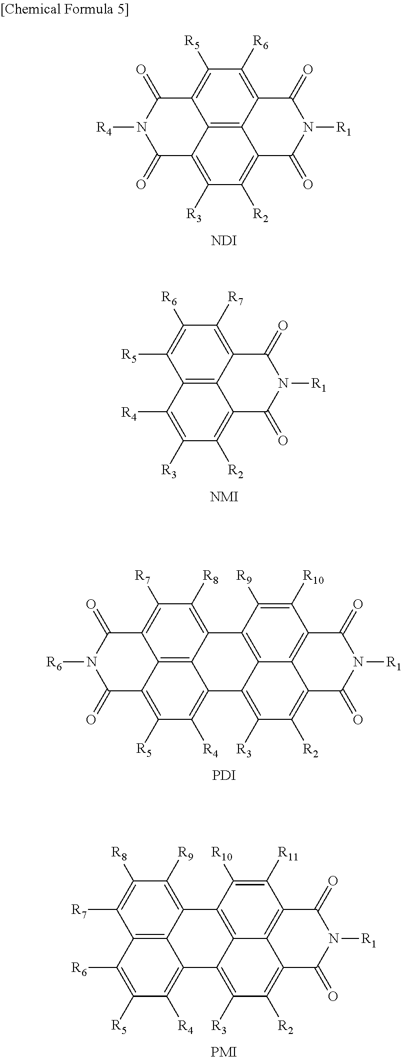

In each formula of [Chemical Formula 5], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group. Any adjacent Rs may be bonded to each other to form a fused aliphatic ring or a fused aromatic ring.

### EXAMPLE

### Example 1

As described above, the photoelectric conversion element according to the embodiment of the present technology is the first electrode and the second electrode arranged to face each other and is provided between the first electrode and the second electrode. The photoelectric conversion element according to the embodiment of the present technology has an organic photoelectric conversion layer including the organic semiconductor that performs photoelectric conversion. The organic photoelectric conversion layer is configured of at least the first organic semiconductor material and the second organic semiconductor material, but may include a third organic semiconductor material. The organic photoelectric conversion layer is configured of a p-type organic semiconductor material and an n-type organic semiconductor material. The organic photoelectric conversion layer photoelectrically converts light of a selective wavelength while transmitting light of other wavelength regions.

Since the hole mainly conducts the p-type semiconductor after the photoelectric conversion, the p-type organic semiconductor material is desirably a material with high hole mobility. In the present technology, the p-type organic semiconductor material (first organic semiconductor material or second organic semiconductor material) included in the organic photoelectric conversion element includes an organic semiconductor having the D₃/Dₜₒₜ of 0.01 or more.

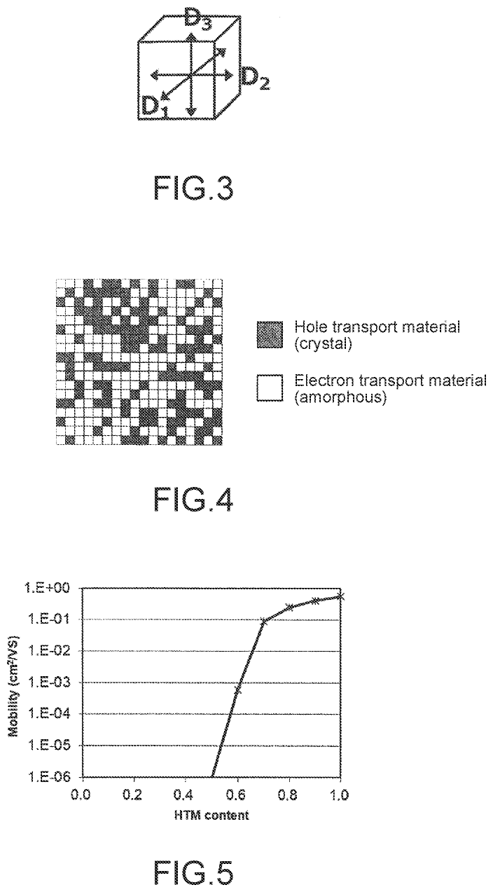

FIG. 3 is a diagram showing an anisotropy of diffusion coefficients in the microcrystal. D₁, D₂, and D₃ are diffusion coefficients Dₓ, D_{y}, and D_{z} in the x-axis, y-axis, and z-axis directions orthogonal to each other in ascending order, with D₃ being the smallest. The D₃/Dₜₒₜ is defined by the equation D₃/Dₜₒₜ=D₃/(D₁+D₂+D₃) . . . (A) using the diffusion coefficients D₁, D₂, and D₃ of the microcrystal shown in FIG. 3.

In the equation (A), the D₁, D₂, and D₃ are the diffusion coefficients Dₓ, D_{y}, and D_{z} in the x-axis, y-axis, and z-axis directions of the microcrystal in ascending order, with D₃ being the smallest. The D₁, D₂, and D₃ can be obtained by a first-principles calculation given a molecule and a crystal structure.

Next, the present inventor determines the mobility of bulk hetero by a roughening kMC method [Non-patent Literature 1] using the D₁, D₂, and D₃.

FIG. 4 is a diagram showing modeling of the bulk hetero layer used in the coarse-grained kMC method. As shown in FIG. 4, the present inventor performs a simulation in a binary system of a hole transport material and an amorphous electron transport material of the crystal. The present inventor three-dimensionally and randomly arranges two types of cells in 100×100×100 cells in accordance with a composition ratio, and performs the simulation under periodic system boundary conditions. A crystal orientation is also randomly arranged for the hole transport material. The hole is generated only on the hole transport material, the hole passes only the hole transport material upon hole diffusion, and the electron transport material is assumed to be impossible to pass.

The present inventor performs the first-principles calculations on pentacene single crystal, which is a typical high-mobility organic semiconductor, to determine the diffusion coefficients D₁, D₂, and D₃. The calculation is performed by a density functional method, and a functional B3LYP and a basis function 6-31++G (d,p) are used. The results are shown in Table 1.

TABLE 1
Diffusion coefficient of pentacene single crystal

| Material | D₁ (cm²/s) | D₂ (cm²/s) | D₃ (cm²/s) | Dₜₒₜ (cm²/s) |
|---|---|---|---|---|
| Pentacene | 2.84E−02 | 2.01E−02 | 1.24E−08 | 4.85E−02 |

In addition, a relationship between the composition ratio and the mobility of the bulk hetero layer determined by the coarse-grained kMC method using these diffusion coefficients is shown in FIG. 5. FIG. 5 is a graph showing a relationship between the composition ratio (horizontal axis) and the mobility (vertical axis) of pentacene.

### Example 2

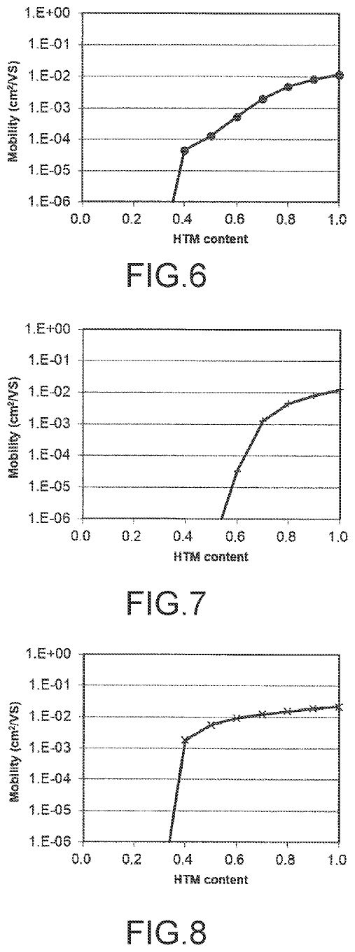

The present inventor determines the diffusion coefficient of the single crystal, and the relationship between the composition of the bulk hetero layer and the hole mobility for rubrene in the same manner as in Example 1. Table 2 shows the diffusion coefficient, and FIG. 6 shows the relationship between the composition ratio and mobility. FIG. 6 is a graph showing the relationship between the composition ratio (horizontal axis) and the mobility (vertical axis) of rubrene.

TABLE 2
Diffusion coefficient of rubrene single crystal

| Material | D₁ (cm²/s) | D₂ (cm²/s) | D₃ (cm²/s) | Dₜₒₜ (cm²/s) |
|---|---|---|---|---|
| Rubrene | 8.99E−02 | 2.62E−04 | 5.67E−07 | 9.01E−02 |

### Example 3

In the same manner as in Example 1, the diffusion coefficient of the single crystal, and the relationship between the composition of the bulk hetero layer and the hole mobility are determined for C₈-BTBT. Table 3 shows the diffusion coefficient, and FIG. 7 shows the relationship between the composition ratio and mobility. FIG. 7 is a graph showing the relationship between the composition ratio (horizontal axis) and the mobility (vertical axis) of C₈-BTBT.

TABLE 3
Diffusion coefficient of C₈-BTBT single crystal

| Material | D₁ (cm²/s) | D₂ (cm²/s) | D₃ (cm²/s) | Dₜₒₜ (cm²/s) |
|---|---|---|---|---|
| C₈-BTBT | 6.70E−03 | 1.33E−04 | 3.32E−09 | 6.83E−03 |

### Example 4

In the same manner as in Example 1, the diffusion coefficient of the single crystal, and the relationship between the composition of the bulk hetero layer and the hole mobility are determined for DPh-BTBT. Table 4 shows the diffusion coefficient, and FIG. 8 shows the relationship between the composition ratio and mobility. FIG. 8 is a graph showing the relationship between the composition ratio (horizontal axis) and the mobility (vertical axis) of DPh-BTBT.

TABLE 4
Diffusion coefficient of DPh-BTBT single crystal

| Material | D₁ (cm²/s) | D₂ (cm²/s) | D₃ (cm²/s) | Dₜₒₜ (cm²/s) |
|---|---|---|---|---|
| DPh-BTBT | 3.87E−03 | 2.94E−04 | 5.15E−05 | 4.22E−03 |

### Example 5

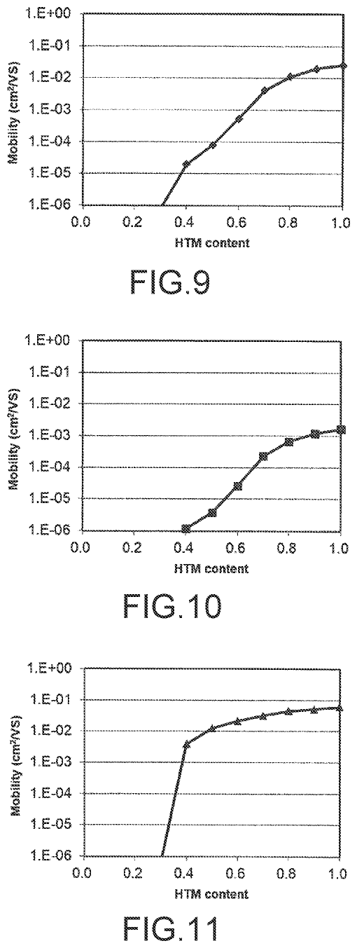

In the same manner as in Example 1, the diffusion coefficient of the single crystal, and the relationship between the composition of the bulk hetero layer and the hole mobility are determined for α-QD. Table 5 shows the diffusion coefficient, and FIG. 9 shows the relationship between the composition ratio and mobility. FIG. 9 is a graph showing the relationship between the composition ratio (horizontal axis) and the mobility (vertical axis) of α-QD.

TABLE 5
Diffusion coefficient of α-QD single crystal

| Material | D₁ (cm²/s) | D₂ (cm²/s) | D₃ (cm²/s) | Dₜₒₜ (cm²/s) |
|---|---|---|---|---|
| α-QD | 1.75E−03 | 1.26E−03 | 3.61E−07 | 3.01E−03 |

### Example 6

In the same manner as in Example 1, the diffusion coefficient of the single crystal, and the relationship between the composition of the bulk hetero layer and the hole mobility are determined for α-QD. Table 6 shows the diffusion coefficient, and FIG. 10 shows the relationship between the composition ratio and mobility. FIG. 10 is a graph showing the relationship between the composition ratio (horizontal axis) and the mobility (vertical axis) of β-QD.

TABLE 6
Diffusion coefficient of β-QD single crystal

| Material | D₁ (cm²/s) | D₂ (cm²/s) | D₃ (cm²/s) | Dₜₒₜ (cm²/s) |
|---|---|---|---|---|
| β-QD | 1.74E−02 | 3.90E−05 | 9.29E−09 | 1.74E−02 |

### Example 7

In the same manner as in Example 1, the diffusion coefficient of the single crystal, and the relationship between the composition of the bulk hetero layer and the hole mobility are determined for γ-QD. Table 7 shows the diffusion coefficient, and FIG. 11 shows the relationship between the composition ratio and mobility. FIG. 11 is a graph showing the relationship between the composition ratio (horizontal axis) and the mobility (vertical axis) of γ-QD.

TABLE 7
Diffusion coefficient of γ-QD single crystal

| Material | D₁ (cm²/s) | D₂ (cm²/s) | D₃ (cm²/s) | Dₜₒₜ (cm²/s) |
|---|---|---|---|---|
| γ-QD | 2.74E−03 | 1.95E−03 | 8.42E−05 | 4.77E−03 |

(Comparison)

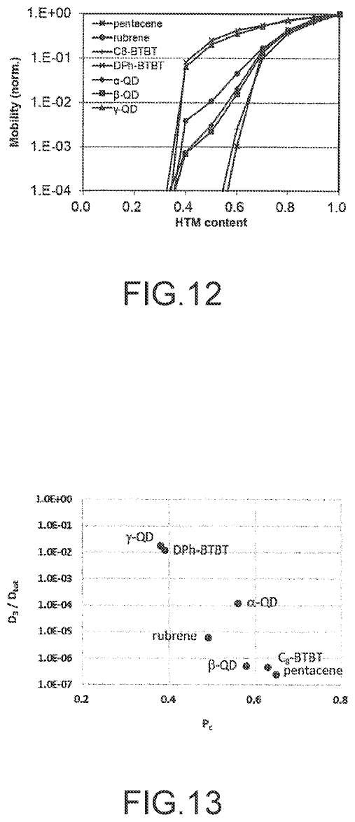

FIG. 12 shows a diagram in which the mobility obtained in Examples 1 to 7 is overwritten. FIG. 12 is a graph showing the relationship between the composition ratio (horizontal axis) and the mobility (vertical axis) of each material. In order to compare Examples 1 to 7, the mobility (vertical axis) in FIG. 12 is normalized by a value of composition=1.0. As a characteristic common to each material, there is a composition in which the mobility decreases drastically. This is a limit point at which a carrier conduction network is interrupted and carriers cannot move smoothly, that is, a percolation limit. Here, the mobility of 1/100 is defined as the percolation limit (P_{c}). Values of the P_{c} and the D₃/Dₜₒₜ of the various materials are shown in Table 8. The relationship between the D₃/Dₜₒₜ and the P_{c} is plotted as shown in FIG. 13. FIG. 13 is a graph showing the relationship between the D₃/Dₜₒₜ (vertical axis) and the P_{c} (horizontal axis).

TABLE 8
P_{c} and D₃/Dₜₒₜ of each material

| Material | D₃/Dₜₒₜ | P_{c} |
|---|---|---|
| Pentacene | 2.56E−07 | 0.65 |
| Rubrene | 6.29E−06 | 0.49 |
| C₈-BTBT | 4.86E−07 | 0.63 |
| DPh-BTBT | 1.22E−02 | 0.39 |
| α-QD | 1.20E−04 | 0.56 |
| β-QD | 5.33E−07 | 0.58 |
| γ-QD | 1.76E−02 | 0.38 |

The composition ratio of the hole transport material in the bulk hetero layer is often 0.4 to 0.6. This is because if the composition ratio is too small, the hole transport material reaches the percolation limit or less and the hole mobility is extremely lowered. Conversely, if the composition ratio of the hole transporting material is too large, the electron transporting material reaches the percolation limit or less and the electron mobility is extremely lowered. If the material whose P_{c} is smaller than the compositional ratio of the hole transporting material is used, higher hole mobility can be obtained in the bulk hetero layer. That is, the material having the P_{c} of 0.4 or less may be used. From FIG. 13, it can be seen that, for the material having the D₃/Dₜₒₜ of 0.01 or more, the P_{c} becomes 0.4 or less. That is, since the percolation limit can be lowered to be equal to or lower than the composition ratio of the hole transporting material, it is possible to have high hole mobility even in the bulk hetero layer. Therefore, in the photoelectric conversion element having the organic photoelectric conversion layer including the organic semiconductor shown in the present examples, the high mobility can be obtained, and excellent afterimage characteristics can be obtained.

### OTHER EMBODIMENTS

As described above, although the present technology have been described by way of embodiments and examples, the discussion and drawings that form a part of this disclosure are not to be understood as limiting the technology. Various alternative embodiments, examples, and operating technology will be apparent to those skilled in the art from this disclosure. The technical scope of the present technology is defined only by matters to specify the invention in the scope of claims that are appropriate from the above description.

<Application Example to Imaging Element>

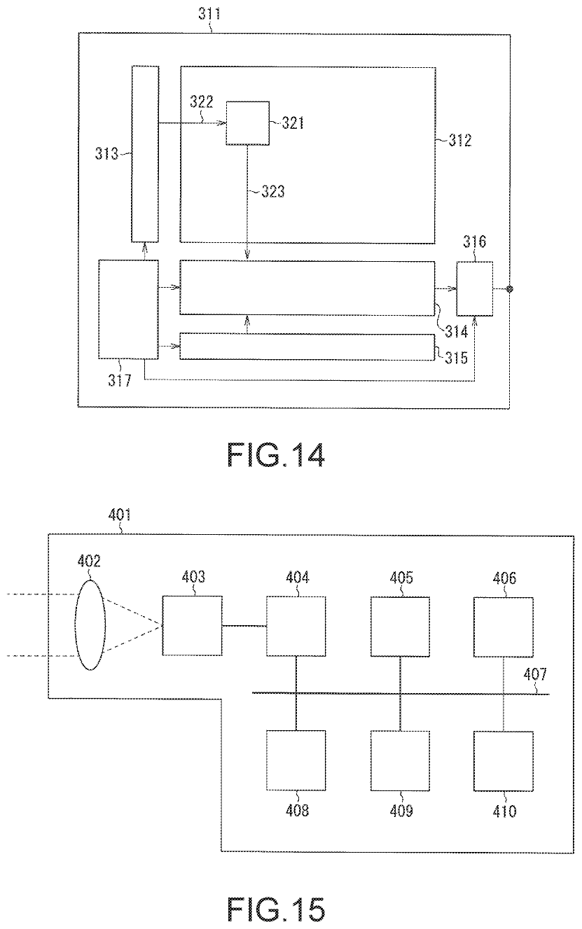

FIG. 14 is a block diagram showing an example of a schematic configuration of an imaging element to which the technology according to the present disclosure (present technology) can be applied.

As shown in FIG. 14, an imaging element 311 is a CMOS type solid-state imaging element, and is configured to include a pixel array section 312, a vertical driving section 313, a column processing section 314, a horizontal driving section 315, an output section 316, and a driving control section 317.

The pixel array section 312 has a plurality of pixels 321 arranged in an array, is connected to the vertical driving section 313 via a plurality of horizontal wires 322 corresponding to the number of rows of the pixels 321, and is connected to the column processing section 314 via a plurality of vertical wires 323 corresponding to the number of columns of the pixels 321. That is, the plurality of pixels 321 included in the pixel array section 312 is arranged at respective points where the horizontal wires 322 and the vertical wires 323 intersect. As the pixel 321, for example, the above-described photoelectric conversion element 10 (see FIGS. 1 and 2) is used.

The vertical driving section 313 sequentially supplies driving signals for driving the respective pixels 321 (transfer signal, selection signal, reset signal, etc.) via the horizontal wires 322 for each row of the plurality of pixels 321 included in the pixel array section 312.

The column processing section 314 extracts a signal level of a pixel signal by performing CDS (Correlated Double Sampling) process on the pixel signal output from the respective pixels 321 via the vertical lines 323 and acquire pixel data corresponding to an amount of received light of the pixels 321.

The horizontal driving section 315 sequentially supplies driving signals with the column processing section 314 for outputting the pixel data acquired from the respective pixels 321 in order from the column processing section 314 for each column of the plurality of pixels 321 included in the pixel array section 312.

The output section 316 is supplied with the pixel data from the column processing section 314 at a timing according to the driving signals of the horizontal driving section 315, amplifies, for example, the pixel data, and outputs to an image processing circuit of the subsequent stage.

The driving control section 317 controls the driving of each block inside the imaging element 311. For example, the driving control section 317 generates a clock signal according to a driving period of each block, and supplies with the respective blocks.

<Application Example to Electronic Apparatus>

The above-described imaging element 311 is applicable to various electronic apparatuses including an imaging system such as a digital still camera and a digital video camera, a mobile phone having an imaging function, and other apparatuses having an imaging function, for example.

FIG. 15 is a block diagram showing a configuration example of an imaging element mounted on the electronic apparatus.

As shown in FIG. 15, an imaging apparatus 401 includes an optical system 402, an imaging element 403, and a DSP (Digital Signal Processor) 404, is configured by connecting the DSP 404, a display apparatus 405, an operation system 406, a memory 408, a recording apparatus 409, and a power supply system 410 via a bus 407, and is capable of capturing still images and moving images.

The optical system 402 is configured to have one or a plurality of lenses, guides image light (incident light) from a subject to the imaging element 403, and images the light receiving surface (sensor section) of the imaging element 403.

As the imaging element 403, the above-described imaging element 311 is applied. Electrons are accumulated on the imaging element 403 in accordance with the image to be imaged on the light receiving surface via the optical system 402 for a constant period. Then, a signal corresponding to the electrons accumulated on the imaging element 403 is supplied to the DSP 404.

The DSP 404 acquires an image by performing various signal processes on the signal from the imaging element 403 and temporarily stores the data of the image in the memory 408. The data of the image stored in the memory 408 is recorded in the recording apparatus 409 or supplied to the display apparatus 405 to display the image. Furthermore, the operation system 406 receives various operations by a user and supplies an operation signal with each block of the imaging apparatus 401, and the power supply system 410 supplies electric power necessary for driving each block of the imaging apparatus 401.

<Application Example to Endoscopic Surgery Systems>

The technology according to the present disclosure (the present technology) is applicable to various products. For example, the technology according to the present disclosure may be applied to an endoscopic surgical system.

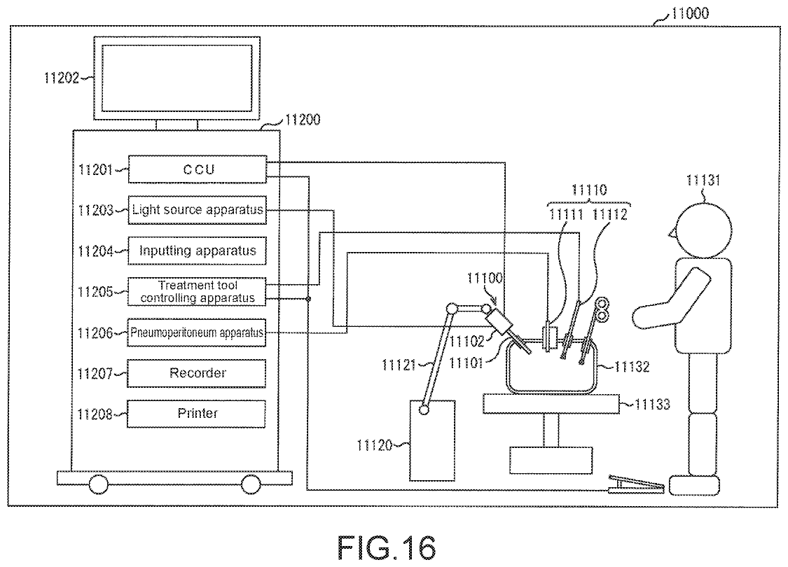

FIG. 16 is a view depicting an example of a schematic configuration of an endoscopic surgery system to which the technology according to an embodiment of the present disclosure (present technology) can be applied.

In FIG. 16, a state is illustrated in which a surgeon (medical doctor) 11131 is using an endoscopic surgery system 11000 to perform surgery for a patient 11132 on a patient bed 11133. As depicted, the endoscopic surgery system 11000 includes an endoscope 11100, other surgical tools 11110 such as a pneumoperitoneum tube 11111 and an energy treatment tool 11112, a supporting arm apparatus 11120 which supports the endoscope 11100 thereon, and a cart 11200 on which various apparatus for endoscopic surgery are mounted.

The endoscope 11100 includes a lens barrel 11101 having a region of a predetermined length from a distal end thereof to be inserted into a body lumen of the patient 11132, and a camera head 11102 connected to a proximal end of the lens barrel 11101. In the example depicted, the endoscope 11100 is depicted which includes as a hard mirror having the lens barrel 11101 of the hard type. However, the endoscope 11100 may otherwise be included as a soft mirror having the lens barrel 11101 of the soft type.

The lens barrel 11101 has, at a distal end thereof, an opening in which an objective lens is fitted. A light source apparatus 11203 is connected to the endoscope 11100 such that light generated by the light source apparatus 11203 is introduced to a distal end of the lens barrel 11101 by a light guide extending in the inside of the lens barrel 11101 and is irradiated toward an observation target in a body lumen of the patient 11132 through the objective lens. It is to be noted that the endoscope 11100 may be a direct view mirror or may be a perspective view mirror or a side view mirror.

An optical system and an image pickup element are provided in the inside of the camera head 11102 such that reflected light (observation light) from the observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 11201.

The CCU 11201 includes a central processing unit (CPU), a graphics processing unit (GPU) or the like and integrally controls operation of the endoscope 11100 and a display apparatus 11202. Further, the CCU 11201 receives an image signal from the camera head 11102 and performs, for the image signal, various image processes for displaying an image based on the image signal such as, for example, a development process (demosaic process).

The display apparatus 11202 displays thereon an image based on an image signal, for which the image processes have been performed by the CCU 11201, under the control of the CCU 11201.

The light source apparatus 11203 includes a light source such as, for example, a light emitting diode (LED) and supplies irradiation light upon imaging of a surgical region to the endoscope 11100.

An inputting apparatus 11204 is an input interface for the endoscopic surgery system 11000. A user can perform inputting of various kinds of information or instruction inputting to the endoscopic surgery system 11000 through the inputting apparatus 11204. For example, the user would input an instruction or a like to change an image pickup condition (type of irradiation light, magnification, focal distance or the like) by the endoscope 11100.

A treatment tool controlling apparatus 11205 controls driving of the energy treatment tool 11112 for cautery or incision of a tissue, sealing of a blood vessel or the like. A pneumoperitoneum apparatus 11206 feeds gas into a body lumen of the patient 11132 through the pneumoperitoneum tube 11111 to inflate the body lumen in order to secure the field of view of the endoscope 11100 and secure the working space for the surgeon. A recorder 11207 is an apparatus capable of recording various kinds of information relating to surgery. A printer 11208 is an apparatus capable of printing various kinds of information relating to surgery in various forms such as a text, an image or a graph.

It is to be noted that the light source apparatus 11203 which supplies irradiation light when a surgical region is to be imaged to the endoscope 11100 may include a white light source which includes, for example, an LED, a laser light source or a combination of them. Where a white light source includes a combination of red, green, and blue (RGB) laser light sources, since the output intensity and the output timing can be controlled with a high degree of accuracy for each color (each wavelength), adjustment of the white balance of a picked up image can be performed by the light source apparatus 11203. Further, in this case, if laser beams from the respective RGB laser light sources are irradiated time-divisionally on an observation target and driving of the image pickup elements of the camera head 11102 are controlled in synchronism with the irradiation timings. Then images individually corresponding to the R, G and B colors can be also picked up time-divisionally. According to this method, a color image can be obtained even if color filters are not provided for the image pickup element.

Further, the light source apparatus 11203 may be controlled such that the intensity of light to be outputted is changed for each predetermined time. By controlling driving of the image pickup element of the camera head 11102 in synchronism with the timing of the change of the intensity of light to acquire images time-divisionally and synthesizing the images, an image of a high dynamic range free from underexposed blocked up shadows and overexposed highlights can be created.

Further, the light source apparatus 11203 may be configured to supply light of a predetermined wavelength band ready for special light observation. In special light observation, for example, by utilizing the wavelength dependency of absorption of light in a body tissue to irradiate light of a narrow band in comparison with irradiation light upon ordinary observation (namely, white light), narrow band observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 11203 can be configured to supply such narrow-band light and/or excitation light suitable for special light observation as described above.

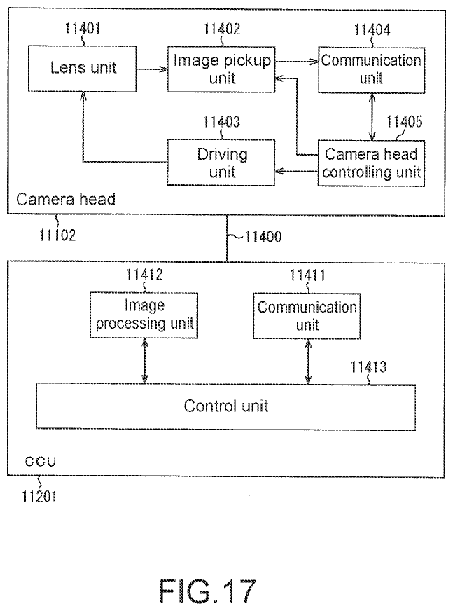

FIG. 17 is a block diagram depicting an example of a functional configuration of the camera head 11102 and the CCU 11201 depicted in FIG. 16.

The camera head 11102 includes a lens unit 11401, an image pickup unit 11402, a driving unit 11403, a communication unit 11404 and a camera head controlling unit 11405. The CCU 11201 includes a communication unit 11411, an image processing unit 11412 and a control unit 11413. The camera head 11102 and the CCU 11201 are connected for communication to each other by a transmission cable 11400.

The lens unit 11401 is an optical system, provided at a connecting location to the lens barrel 11101. Observation light taken in from a distal end of the lens barrel 11101 is guided to the camera head 11102 and introduced into the lens unit 11401. The lens unit 11401 includes a combination of a plurality of lenses including a zoom lens and a focusing lens.

The number of image pickup elements which is included by the image pickup unit 11402 may be one (single-plate type) or a plural number (multi-plate type). Where the image pickup unit 11402 is configured as that of the multi-plate type, for example, image signals corresponding to respective R, G and B are generated by the image pickup elements, and the image signals may be synthesized to obtain a color image. The image pickup unit 11402 may also be configured so as to have a pair of image pickup elements for acquiring respective image signals for the right eye and the left eye ready for three dimensional (3D) display. If 3D display is performed, then the depth of a living body tissue in a surgical region can be comprehended more accurately by the surgeon 11131. It is to be noted that, where the image pickup unit 11402 is configured as that of stereoscopic type, a plurality of systems of lens units 11401 are provided corresponding to the individual image pickup elements.

Further, the image pickup unit 11402 may not necessarily be provided on the camera head 11102. For example, the image pickup unit 11402 may be provided immediately behind the objective lens in the inside of the lens barrel 11101.

The driving unit 11403 includes an actuator and moves the zoom lens and the focusing lens of the lens unit 11401 by a predetermined distance along an optical axis under the control of the camera head controlling unit 11405. Consequently, the magnification and the focal point of a picked up image by the image pickup unit 11402 can be adjusted suitably.

The communication unit 11404 includes a communication apparatus for transmitting and receiving various kinds of information to and from the CCU 11201. The communication unit 11404 transmits an image signal acquired from the image pickup unit 11402 as RAW data to the CCU 11201 through the transmission cable 11400.

In addition, the communication unit 11404 receives a control signal for controlling driving of the camera head 11102 from the CCU 11201 and supplies the control signal to the camera head controlling unit 11405. The control signal includes information relating to image pickup conditions such as, for example, information that a frame rate of a picked up image is designated, information that an exposure value upon image picking up is designated and/or information that a magnification and a focal point of a picked up image are designated.

It is to be noted that the image pickup conditions such as the frame rate, exposure value, magnification or focal point may be designated by the user or may be set automatically by the control unit 11413 of the CCU 11201 on the basis of an acquired image signal. In the latter case, an auto exposure (AE) function, an auto focus (AF) function and an auto white balance (AWB) function are incorporated in the endoscope 11100.

The camera head controlling unit 11405 controls driving of the camera head 11102 on the basis of a control signal from the CCU 11201 received through the communication unit 11404.

The communication unit 11411 includes a communication apparatus for transmitting and receiving various kinds of information to and from the camera head 11102. The communication unit 11411 receives an image signal transmitted thereto from the camera head 11102 through the transmission cable 11400.

Further, the communication unit 11411 transmits a control signal for controlling driving of the camera head 11102 to the camera head 11102. The image signal and the control signal can be transmitted by electrical communication, optical communication or the like.

The image processing unit 11412 performs various image processes for an image signal in the form of RAW data transmitted thereto from the camera head 11102.

The control unit 11413 performs various kinds of control relating to image picking up of a surgical region or the like by the endoscope 11100 and display of a picked up image obtained by image picking up of the surgical region or the like. For example, the control unit 11413 creates a control signal for controlling driving of the camera head 11102.

Further, the control unit 11413 controls, on the basis of an image signal for which image processes have been performed by the image processing unit 11412, the display apparatus 11202 to display a picked up image in which the surgical region or the like is imaged. Thereupon, the control unit 11413 may recognize various objects in the picked up image using various image recognition technologies. For example, the control unit 11413 can recognize a surgical tool such as forceps, a particular living body region, bleeding, mist when the energy treatment tool 11112 is used and so forth by detecting the shape, color and so forth of edges of objects included in a picked up image. The control unit 11413 may cause, when it controls the display apparatus 11202 to display a picked up image, various kinds of surgery supporting information to be displayed in an overlapping manner with an image of the surgical region using a result of the recognition. Where surgery supporting information is displayed in an overlapping manner and presented to the surgeon 11131, the burden on the surgeon 11131 can be reduced and the surgeon 11131 can proceed with the surgery with certainty.

The transmission cable 11400 which connects the camera head 11102 and the CCU 11201 to each other is an electric signal cable ready for communication of an electric signal, an optical fiber ready for optical communication or a composite cable ready for both of electrical and optical communications.

Here, while, in the example depicted, communication is performed by wired communication using the transmission cable 11400, the communication between the camera head 11102 and the CCU 11201 may be performed by wireless communication.

An example of the endoscopic surgery system to which the technology according to the present disclosure can be applied is described above. The technology according to the present disclosure can be applied to, for example, the endoscopes 11100, the image pickup unit 11402 of the camera head 11102, the image processing unit 11412 of the CCU 11201, and the like among the above-described configurations. Specifically, the photoelectric conversion element 10 shown in FIGS. 1 and 2, or the imaging element 311 shown in FIG. 14, can be applied to the image pickup unit 11402. By applying the technology according to the present disclosure to the endoscope 11100, the image pickup unit 11402 of the camera head 11102, the image processor 11412 of the CCU 11201, and the like, a sharper surgical portion image can be obtained, so that the surgeon can surely confirm the surgical portion. In addition, by applying the technology according to the present disclosure to the endoscope 11100, the image pickup unit 11402 of the camera head 11102, the image processing unit 11412 of the CCU 11201, and the like, a surgical portion image can be obtained with a lower latency, so that the surgeon can perform a treatment with the same feeling as when the surgeon performs a tactile observation of the surgical portion.

Note that, although the endoscopic surgical system is described herein as an example, the technology according to the present disclosure may be applied to, for example, a microscope surgical system or the like in addition thereto.

<Application Example to Mobile Body>

The technology according to the present disclosure (present technology) is applicable to various products. For example, the technology according to the present disclosure may be realized as an apparatus mounted on any type of moving objects such as an automobile, an electric vehicle, a hybrid electric vehicle, a motorcycle, a bicycle, personal mobility, an airplane, a drone, a ship, and a robot.

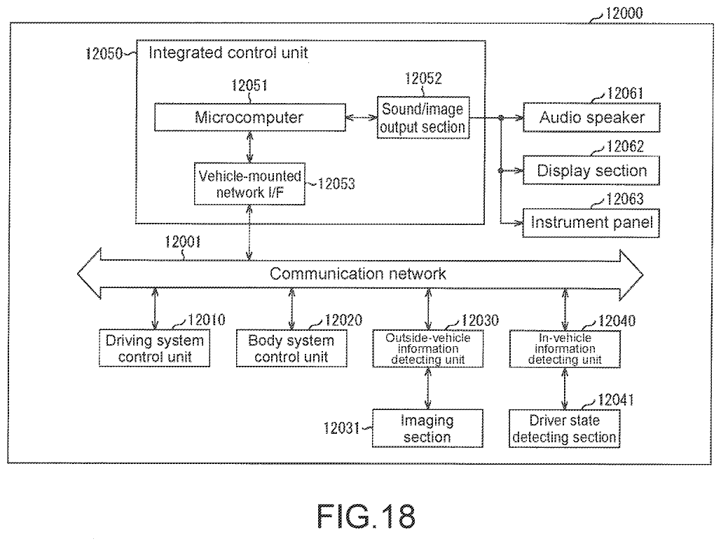

FIG. 18 is a block diagram depicting an example of schematic configuration of a vehicle control system as an example of a mobile body control system to which the technology according to an embodiment of the present disclosure can be applied.

The vehicle control system 12000 includes a plurality of electronic control units connected to each other via a communication network 12001. In the example depicted in FIG. 18, the vehicle control system 12000 includes a driving system control unit 12010, a body system control unit 12020, an outside-vehicle information detecting unit 12030, an in-vehicle information detecting unit 12040, and an integrated control unit 12050. In addition, a microcomputer 12051, a sound/image output section 12052, and a vehicle-mounted network interface (I/F) 12053 are illustrated as a functional configuration of the integrated control unit 12050.

The driving system control unit 12010 controls the operation of devices related to the driving system of the vehicle in accordance with various kinds of programs. For example, the driving system control unit 12010 functions as a control device for a driving force generating device for generating the driving force of the vehicle, such as an internal combustion engine, a driving motor, or the like, a driving force transmitting mechanism for transmitting the driving force to wheels, a steering mechanism for adjusting the steering angle of the vehicle, a braking device for generating the braking force of the vehicle, and the like.

The body system control unit 12020 controls the operation of various kinds of devices provided to a vehicle body in accordance with various kinds of programs. For example, the body system control unit 12020 functions as a control device for a keyless entry system, a smart key system, a power window device, or various kinds of lamps such as a headlamp, a backup lamp, a brake lamp, a turn signal, a fog lamp, or the like. In this case, radio waves transmitted from a mobile device as an alternative to a key or signals of various kinds of switches can be input to the body system control unit 12020. The body system control unit 12020 receives these input radio waves or signals, and controls a door lock device, the power window device, the lamps, or the like of the vehicle.

The outside-vehicle information detecting unit 12030 detects information about the outside of the vehicle including the vehicle control system 12000. For example, the outside-vehicle information detecting unit 12030 is connected with an imaging section 12031. The outside-vehicle information detecting unit 12030 makes the imaging section 12031 image an image of the outside of the vehicle, and receives the imaged image. On the basis of the received image, the outside-vehicle information detecting unit 12030 may perform processing of detecting an object such as a human, a vehicle, an obstacle, a sign, a character on a road surface, or the like, or processing of detecting a distance thereto.

The imaging section 12031 is an optical sensor that receives light, and which outputs an electric signal corresponding to a received light amount of the light. The imaging section 12031 can output the electric signal as an image, or can output the electric signal as information about a measured distance. In addition, the light received by the imaging section 12031 may be visible light, or may be invisible light such as infrared rays or the like.

The in-vehicle information detecting unit 12040 detects information about the inside of the vehicle. The in-vehicle information detecting unit 12040 is, for example, connected with a driver state detecting section 12041 that detects the state of a driver. The driver state detecting section 12041, for example, includes a camera that images the driver. On the basis of detection information input from the driver state detecting section 12041, the in-vehicle information detecting unit 12040 may calculate a degree of fatigue of the driver or a degree of concentration of the driver, or may determine whether the driver is dozing.

The microcomputer 12051 can calculate a control target value for the driving force generating device, the steering mechanism, or the braking device on the basis of the information about the inside or outside of the vehicle which information is obtained by the outside-vehicle information detecting unit 12030 or the in-vehicle information detecting unit 12040, and output a control command to the driving system control unit 12010. For example, the microcomputer 12051 can perform cooperative control intended to implement functions of an advanced driver assistance system (ADAS) which functions include collision avoidance or shock mitigation for the vehicle, following driving based on a following distance, vehicle speed maintaining driving, a warning of collision of the vehicle, a warning of deviation of the vehicle from a lane, or the like.

In addition, the microcomputer 12051 can perform cooperative control intended for automatic driving, which makes the vehicle to travel autonomously without depending on the operation of the driver, or the like, by controlling the driving force generating device, the steering mechanism, the braking device, or the like on the basis of the information about the outside or inside of the vehicle which information is obtained by the outside-vehicle information detecting unit 12030 or the in-vehicle information detecting unit 12040.

In addition, the microcomputer 12051 can output a control command to the body system control unit 12020 on the basis of the information about the outside of the vehicle which information is obtained by the outside-vehicle information detecting unit 12030. For example, the microcomputer 12051 can perform cooperative control intended to prevent a glare by controlling the headlamp so as to change from a high beam to a low beam, for example, in accordance with the position of a preceding vehicle or an oncoming vehicle detected by the outside-vehicle information detecting unit 12030.

The sound/image output section 12052 transmits an output signal of at least one of a sound and an image to an output device capable of visually or auditorily notifying information to an occupant of the vehicle or the outside of the vehicle. In the example of FIG. 18, an audio speaker 12061, a display section 12062, and an instrument panel 12063 are illustrated as the output device. The display section 12062 may, for example, include at least one of an on-board display and a head-up display.

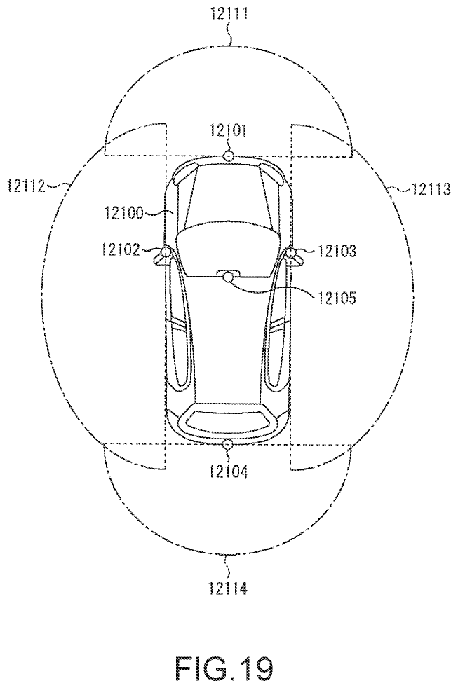

FIG. 19 is a diagram depicting an example of the installation position of the imaging section 12031.

In FIG. 19, the imaging section 12031 includes imaging sections 12101, 12102, 12103, 12104, and 12105.

The imaging sections 12101, 12102, 12103, 12104, and 12105 are, for example, disposed at positions on a front nose, sideview mirrors, a rear bumper, and a back door of the vehicle 12100 as well as a position on an upper portion of a windshield within the interior of the vehicle. The imaging section 12101 provided to the front nose and the imaging section 12105 provided to the upper portion of the windshield within the interior of the vehicle obtain mainly an image of the front of the vehicle 12100. The imaging sections 12102 and 12103 provided to the sideview mirrors obtain mainly an image of the sides of the vehicle 12100. The imaging section 12104 provided to the rear bumper or the back door obtains mainly an image of the rear of the vehicle 12100. The imaging section 12105 provided to the upper portion of the windshield within the interior of the vehicle is used mainly to detect a preceding vehicle, a pedestrian, an obstacle, a signal, a traffic sign, a lane, or the like.

Incidentally, FIG. 19 depicts an example of photographing ranges of the imaging sections 12101 to 12104. An imaging range 12111 represents the imaging range of the imaging section 12101 provided to the front nose. Imaging ranges 12112 and 12113 respectively represent the imaging ranges of the imaging sections 12102 and 12103 provided to the sideview mirrors. An imaging range 12114 represents the imaging range of the imaging section 12104 provided to the rear bumper or the back door. A bird's-eye image of the vehicle 12100 as viewed from above is obtained by superimposing image data imaged by the imaging sections 12101 to 12104, for example.

At least one of the imaging sections 12101 to 12104 may have a function of obtaining distance information. For example, at least one of the imaging sections 12101 to 12104 may be a stereo camera constituted of a plurality of imaging elements, or may be an imaging element having pixels for phase difference detection.

For example, the microcomputer 12051 can determine a distance to each three-dimensional object within the imaging ranges 12111 to 12114 and a temporal change in the distance (relative speed with respect to the vehicle 12100) on the basis of the distance information obtained from the imaging sections 12101 to 12104, and thereby extract, as a preceding vehicle, a nearest three-dimensional object in particular that is present on a traveling path of the vehicle 12100 and which travels in substantially the same direction as the vehicle 12100 at a predetermined speed (for example, equal to or more than 0 km/hour). Further, the microcomputer 12051 can set a following distance to be maintained in front of a preceding vehicle in advance, and perform automatic brake control (including following stop control), automatic acceleration control (including following start control), or the like. It is thus possible to perform cooperative control intended for automatic driving that makes the vehicle travel autonomously without depending on the operation of the driver or the like.

For example, the microcomputer 12051 can classify three-dimensional object data on three-dimensional objects into three-dimensional object data of a two-wheeled vehicle, a standard-sized vehicle, a large-sized vehicle, a pedestrian, a utility pole, and other three-dimensional objects on the basis of the distance information obtained from the imaging sections 12101 to 12104, extract the classified three-dimensional object data, and use the extracted three-dimensional object data for automatic avoidance of an obstacle. For example, the microcomputer 12051 identifies obstacles around the vehicle 12100 as obstacles that the driver of the vehicle 12100 can recognize visually and obstacles that are difficult for the driver of the vehicle 12100 to recognize visually. Then, the microcomputer 12051 determines a collision risk indicating a risk of collision with each obstacle. In a situation in which the collision risk is equal to or higher than a set value and there is thus a possibility of collision, the microcomputer 12051 outputs a warning to the driver via the audio speaker 12061 or the display section 12062, and performs forced deceleration or avoidance steering via the driving system control unit 12010. The microcomputer 12051 can thereby assist in driving to avoid collision.

At least one of the imaging sections 12101 to 12104 may be an infrared camera that detects infrared rays. The microcomputer 12051 can, for example, recognize a pedestrian by determining whether or not there is a pedestrian in imaged images of the imaging sections 12101 to 12104. Such recognition of a pedestrian is, for example, performed by a procedure of extracting characteristic points in the imaged images of the imaging sections 12101 to 12104 as infrared cameras and a procedure of determining whether or not it is the pedestrian by performing pattern matching processing on a series of characteristic points representing the contour of the object. When the microcomputer 12051 determines that there is a pedestrian in the imaged images of the imaging sections 12101 to 12104, and thus recognizes the pedestrian, the sound/image output section 12052 controls the display section 12062 so that a square contour line for emphasis is displayed so as to be superimposed on the recognized pedestrian. The sound/image output section 12052 may also control the display section 12062 so that an icon or the like representing the pedestrian is displayed at a desired position.

An example of the vehicle control system to which the technology according to the present disclosure can be applied is described above. The technology according to the present disclosure can be applied to the imaging section 12031 or the like among the configurations described above. Specifically, the photoelectric conversion element 10 shown in FIGS. 1 and 2, or the imaging element 311 shown in FIG. 14, can be applied to the imaging section 12031. By applying the technology according to the present disclosure to the imaging section 12031, it is possible to obtain a more visible captured image and to thereby reduce the fatigue of the driver.

Note that the present technology may also take the following configurations.

(1) A photoelectric conversion element, including:

a first electrode and a second electrode arranged to face each other; and

a photoelectric conversion layer provided between the first electrode and the second electrode and including a first organic semiconductor material and a second organic semiconductor material, in which

the first organic semiconductor material or the second organic semiconductor material includes an organic semiconductor having a D₃/Dₜₒₜ of 0.01 or more.

(2) The photoelectric conversion element according to (1), in which the first organic semiconductor material or the second organic semiconductor material includes an organic semiconductor having a P_{c} of 0.4 or less.
(3) The photoelectric conversion element according to (1) or (2), in which the photoelectric conversion layer further includes a third organic semiconductor.
(4) The photoelectric conversion element according to any one of (1) to (3), in which the organic semiconductor is any one or more of the compounds represented by the formulae of [Chemical Formula 6].

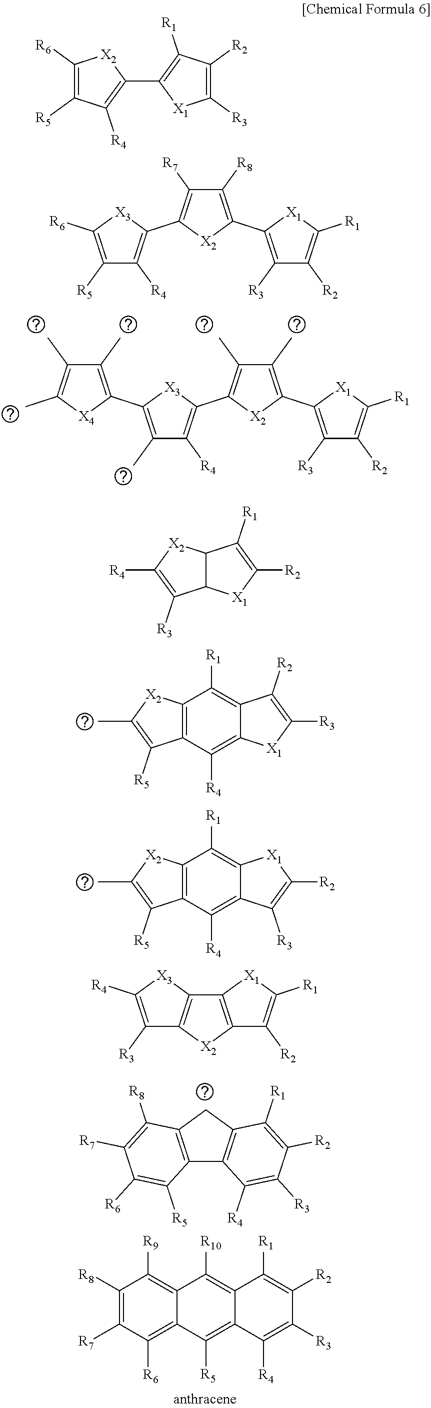

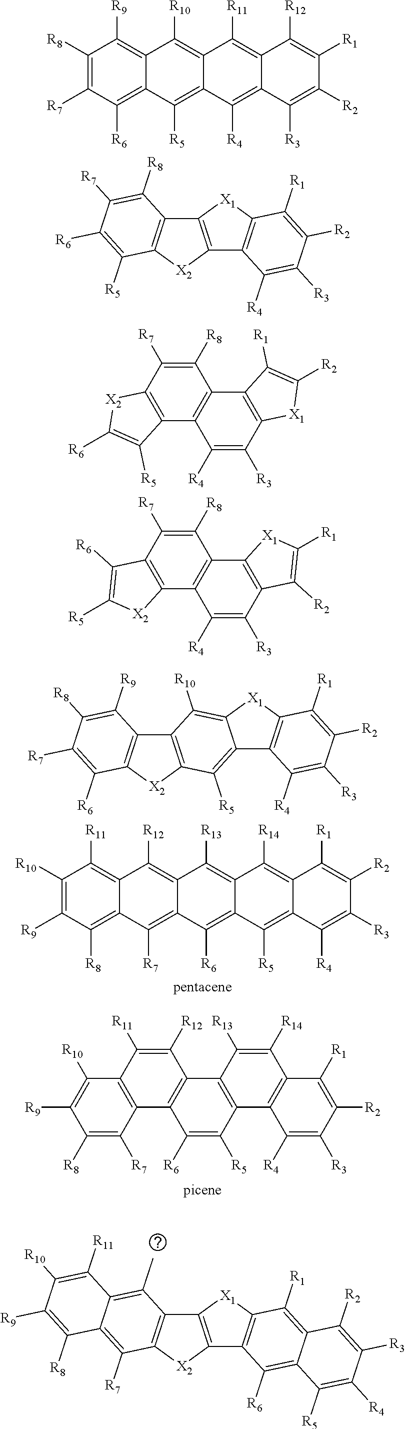

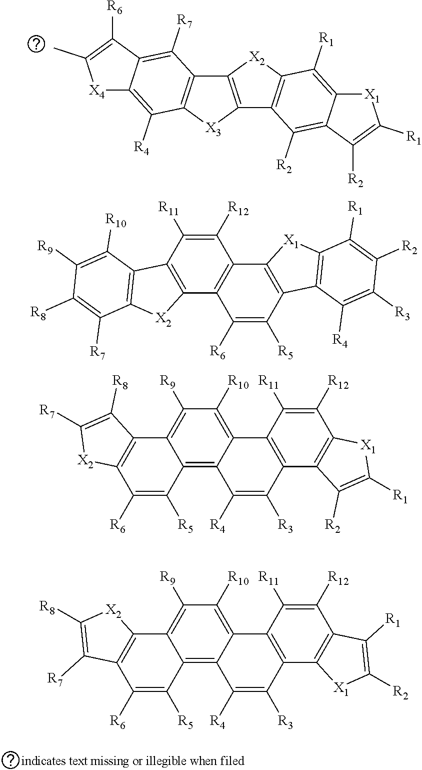

(in each formula of [Chemical Formula 6], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring, and X is each independently a heteroatom).

(5) The photoelectric conversion element according to any one of (1) to (3), in which the organic semiconductor is a compound represented by the formula (1) of [Chemical Formula 7]

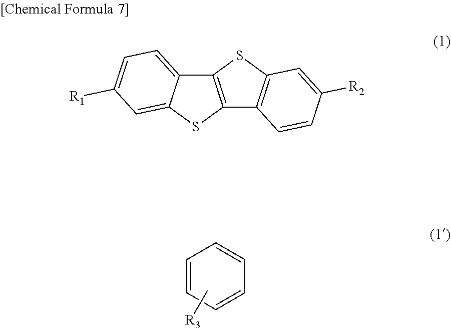

(in the formula (1) of [Formula 7], R1 and R2 are each independently a hydrogen atom or a substituent represented by the formula (1)′, R3 is an aromatic ring group or an aromatic ring group having a substituent).

(6) The photoelectric conversion element according to any one of (1) to (3), the organic semiconductor is a compound represented by the formula of [Chemical Formula 8].

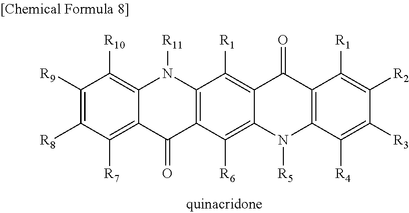

(in the formula of [Chemical Formula 8], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring, and X is each independently a heteroatom).

(7) The photoelectric conversion element according to any one of (1) to (3), in which the organic semiconductor is any one or more of the compounds represented by the formulae [Chemical Formula 9].

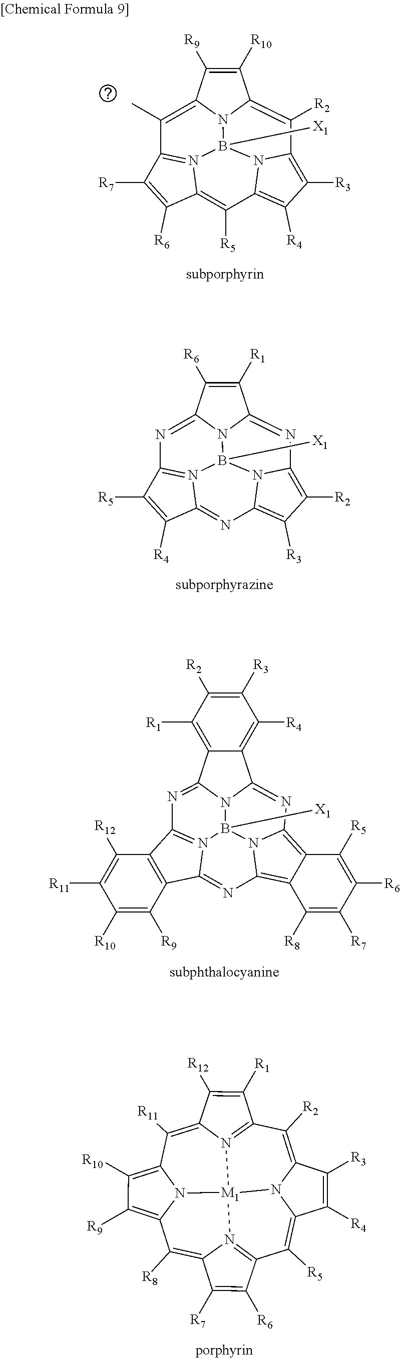

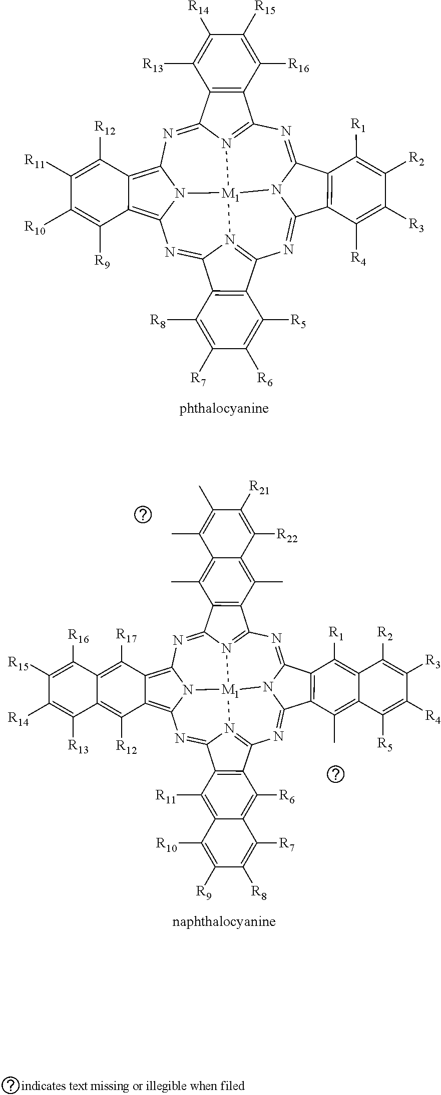

(in each formula of [Chemical Formula 9], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring, X is an anionic group. M is a cationic group).

(8) The optical transducer according to any one of (1) to (3), the organic semiconductor is any one or more of the compounds represented by the formulae [Chemical Formula 10].

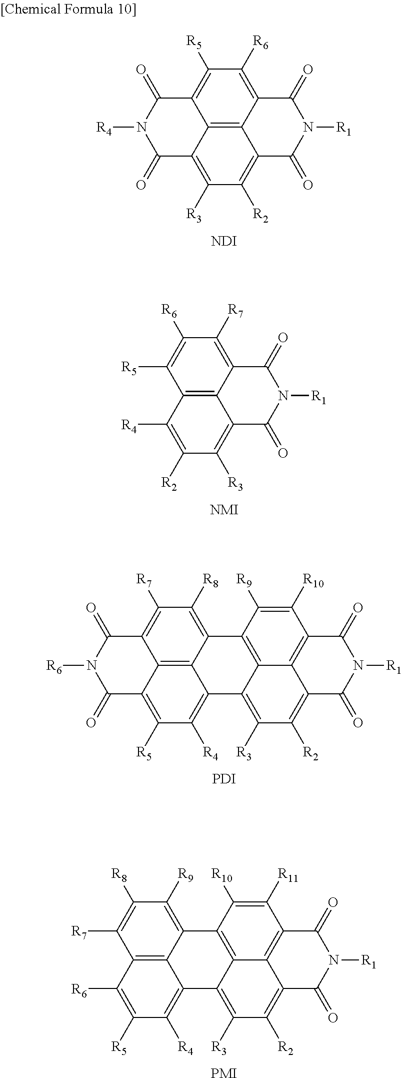

(in each formula of [Chemical Formula 10], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring).

### REFERENCE SIGNS LIST

10 Photoelectric conversion element
11 semiconductor substrate
11B inorganic photoelectric conversion section
11G organic photoelectric conversion section
11R inorganic photoelectric conversion section
12, 52 interlayer insulating film
13a, 13b, 15b wiring layer
14 interlayer insulating film
15a interlayer insulating film
16 insulating film
17 organic photoelectric conversion layer
18 protective layer
19 protective layer
20 contact metal layer
21 planarization layer
22 on-chip lens
51 multilayer interconnection layer
51a wiring
53 supporting substrate
110 silicon layer
110G green storage layer
120a, 20b, 120b, 120b2 conductive plug
211 electrode for charge storage
212 charge storage electrode
215 photoelectric conversion layer
216 second electrode
281 interlayer insulating layer
282 insulating layer
283 protective layer
290 on-chip micro lens
311 imaging element
312 pixel array section
313 vertical driving section
314 column processing section
315 horizontal driving section
316 output section
317 driving control section
321 pixel
322 horizontal wire
323 vertical wire
401 imaging apparatus
402 optical system
403 imaging element
405 display device
406 operation system
407 bus
408 memory
409 recording device
410 power supply system
11000 endoscopic surgery system
11100 endoscope
11101 barrel
11102 camera head
11110 operative instrument
11111 pneumoperitoneum tube
11112 energy treatment apparatus
11120 support arm device
11131 surgeon
11132 patient
11133 patient bed
11200 cart
11201 CCU
11202 display device
11203 light source apparatus
11204 inputting apparatus
11205 treatment tool controlling apparatus
11206 pneumoperitoneum apparatus
11207 recorder
11208 printer
11400 transmission cable
11401 lens unit
11402 image pickup unit
11403 driving unit
11404 communication unit
11405 camera head controlling unit
11411 communication unit
11412 image processing unit
11413 control unit
12000 vehicle control system
12001 communication network
12010 driving system control unit
12020 body system control unit
12030 outside-vehicle information detecting section
12031 imaging section
12040 in-vehicle information detecting unit
12041 driver state detecting section
12050 integrated control unit
12051 microcomputer
12052 sound/image output section
12061 audio speaker
12062 display section
12063 instrument panel
12100 vehicle
12101, 12102, 12103, 12104, 12105 imaging section
12111, 12112, 12113, 12114 imaging range
D1, D2, Dx, Dy, Dz diffusion coefficient
TG1, TG2, TG3 gate electrode

What is claimed is:

1. A photoelectric conversion element, comprising:
a first electrode and a second electrode arranged to face each other; and
a photoelectric conversion layer provided between the first electrode and the second electrode and including a first organic semiconductor material and a second organic semiconductor material, wherein
the first organic semiconductor material or the second organic semiconductor material includes an organic semiconductor having a D₃/Dₜₒₜ of 0.01 or more.

2. The photoelectric conversion element according to claim 1, wherein the first organic semiconductor material or the second organic semiconductor material includes an organic semiconductor having a P_{c} of 0.4 or less.

3. The photoelectric conversion element according to claim 1, wherein the photoelectric conversion layer further includes a third organic semiconductor.

4. The photoelectric conversion element according to claim 1, wherein the organic semiconductor is any one or more of the compounds represented by the formulae of [Chemical Formula 1] 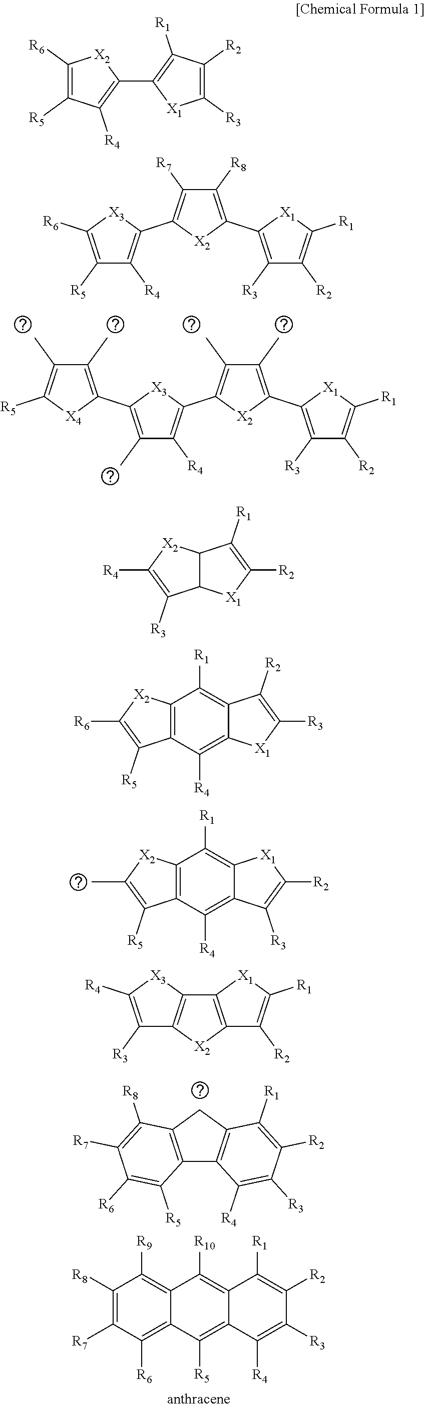 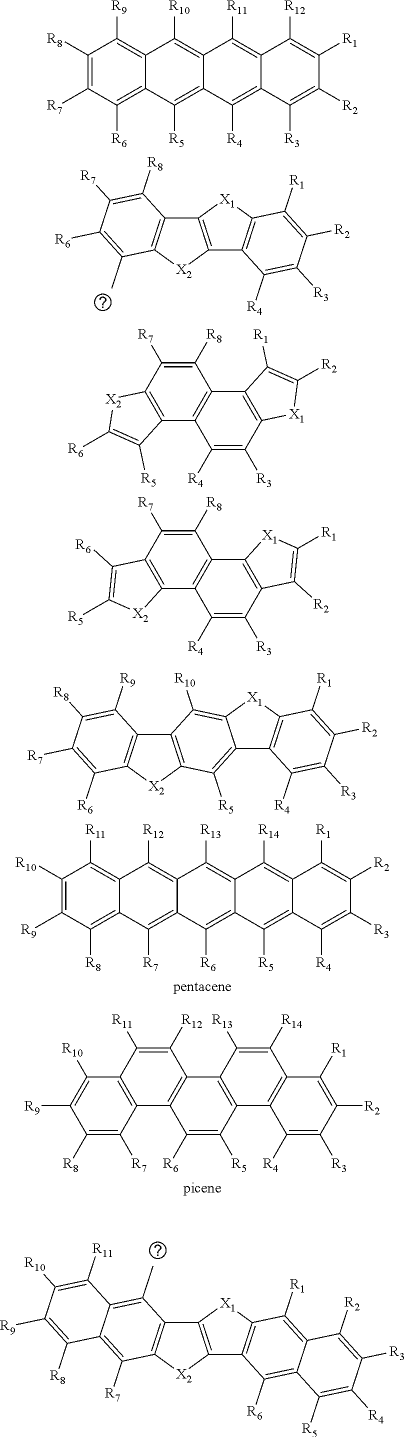 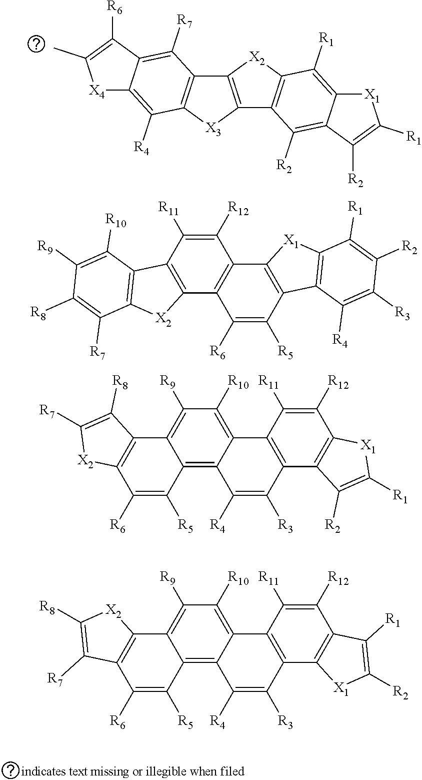
(in each formula of [Chemical Formula 1], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring, and X is each independently a heteroatom).

5. The photoelectric conversion element according to claim 1, wherein the organic semiconductor is a compound represented by the formula (1) of [Chemical Formula 2] 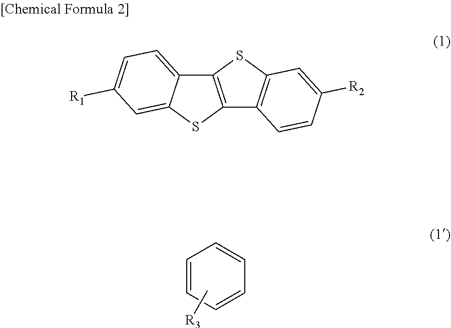
(in the formula (1) of [Chemical Formula 2], R1 and R2 are each independently a hydrogen atom or a substituent represented by the formula (1)′, and R3 is an aromatic ring group or an aromatic ring group having a substituent).

6. The photoelectric conversion element according to claim 1, wherein the organic semiconductor is a compound represented by the formula of [Chemical Formula 3] 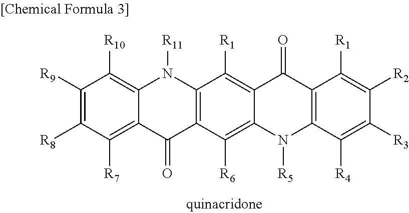
(in the formula of [Chemical Formula 3], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring).

7. The photoelectric conversion element according to claim 1, wherein the organic semiconductor is any one or more of the compounds represented by the formulae of [Chemical Formula 4] 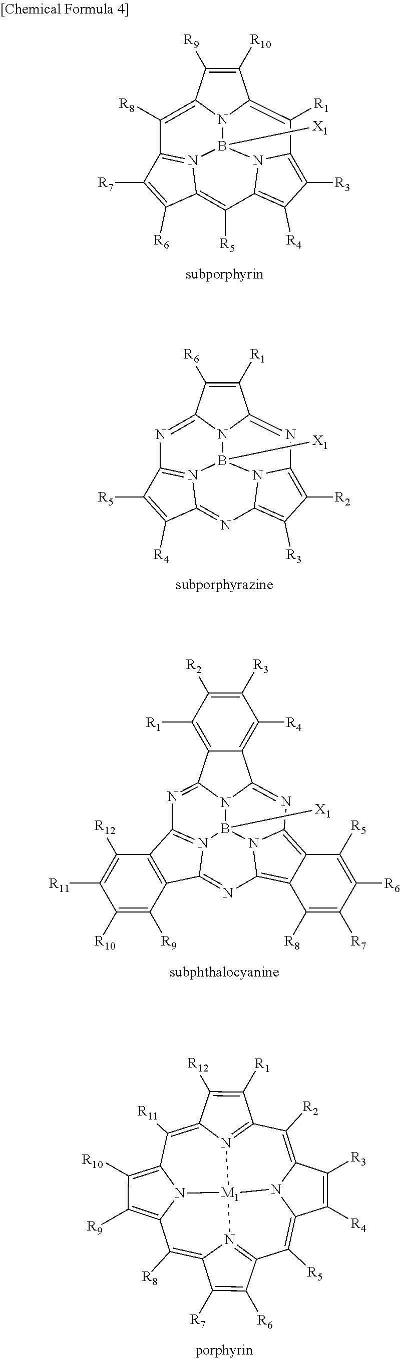 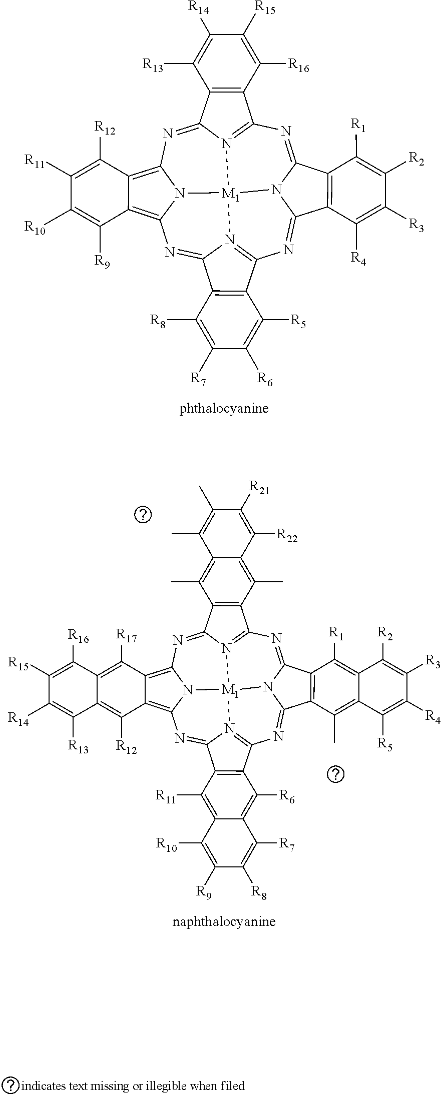
(in each formula of [Chemical Formula 4], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, any adjacent Rs may be bonded to each other to form a fused aliphatic ring or a fused aromatic ring, and X is an anionic group, and M is a cationic group).

8. The optical transducer according to claim 1, wherein the organic semiconductor is any one or more of the compounds represented by the formulae of [Chemical Formula 5] 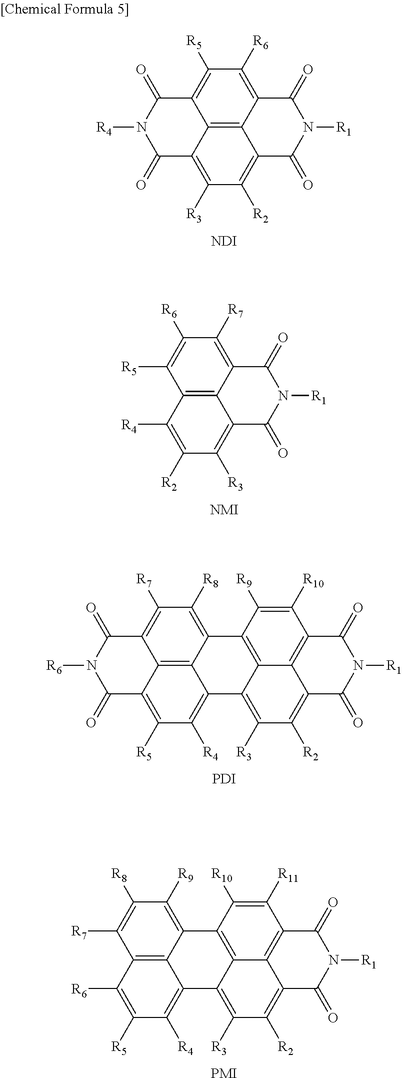
(in each formula of [Chemical Formula 5], R is each independently a hydrogen atom, a halogen atom, a substituted or unsubstituted alkyl group, a substituted or unsubstituted aryl group, a substituted or unsubstituted heteroaryl group, a thioalkyl group, a thioaryl group, an arylsulfonyl group, an alkylsulfonyl group, an amino group, an alkylamino group, an arylamino group, a hydroxy group, an alkoxy group, an acylamino group, an acyloxy group, a carboxy group, a carboxyamide group, a carboxyalkoxy group, an acyl group, a sulfonyl group, a cyano group, and a nitro group, and any adjacent Rs may be bonded to each other to form a fused aliphatic ring or a fused aromatic ring).
